(19) United States
(12) Patent Application Publication
(10) Pub. No.: US 2002/0026659 A1
(43) Pub. Date: Feb. 28, 2002

(54) Developed seed and methods for making the same

(76) Inventors: Alan D. Blowers, St. Charles, IL (US); Robert D. Conrad, Wheaton, IL (US); Kimberly A. Funk, Oswego, IL (US); Zubin Khambatta, Orland Park, IL (US); Audrey Charles, Naperville, IL (US)

Correspondence Address: Edward P. Gamson, Esq., Welsh & Katz, Ltd., Suite 2200, 120 S. Riverside Plaza, Chicago, IL, 60606, US

(21) Appl. No.: 09/750,980

(22) Filed: Dec. 28, 2000

Related U.S. Application Data
(63) Continuation of application No. 09/611,588, filed on Jul. 7, 2000.
Application No. 09/611,588 is a continuation-in-part of application No. 09/611,598, filed on Jul. 7, 2000.
(60) Provisional application No. 60/148,354, filed on Aug. 11, 1999.
(60) Provisional application No. 60/143,016, filed on Jul. 9, 1999.

(51) Int. Cl. A01C 1/06; A01C 21/00; A01H 5/00; C12N 5/00; C12N 5/02

(52) U.S. Cl. 800/298; 435/430; 47/57.6

(57) ABSTRACT

The present invention relates to developed seed that is singulated and free-flowing, and comprises a modified root structure whose root development is interrupted and altered, but that root development is capable of resuming when the developed seed is sown in a suitable environment. The developed seed can exhibit an emerged hypocotyl or lack an emerged hypocotyl. The developed seed is prepared as desiccation-intolerant seed and can be converted to be desiccation-tolerant. The present invention also relates to methods of making the developed seed.

### RELATED APPLICATION INFORMATION

The present application is a continuation-in-part of application Ser. No. 09/611,598, filed on Jul. 7, 2000 that claims priority from U.S. application Ser. No. 60/148,354, filed Aug. 11, 1999, and U.S. application Ser. No. 60/143,016 filed on Jul. 9, 1999.

### FIELD OF THE INVENTION

The present invention relates to developmentally-advanced, developed seed. The developed seed of the present invention is unique in that the normal root development of the developed seed has been interrupted and altered, and root development can be re-initiated and resumed when the developed seed is sown in a suitable environment. The developed seed gives rise to a usable plant. The present invention also relates to methods for making developed seed. The developed seed can exhibit desiccation tolerance or not.

### BACKGROUND OF THE INVENTION

Several attempts have been made to produce pregerminated seed that gives rise to consistently high and reproducible rates of germination in the greenhouse or field for many species of plants. Pregerminated seeds, in which the radicle has emerged, offers the potential advantage of faster germination times once sown. Despite this apparent advantage, the emergence of the radicle (a very early event in the process of seedling development) remains only a rough predictor of the timing and uniformity of hypocotyl and cotyledon development in a young plant. For example, the seeds of some species may germinate relatively fast (as defined by emergence of the radicle), but then exhibit fairly lengthy time periods before hypocotyl and cotyledonary leaf development are evident. In these examples, the advantages offered by pregerminated seed are largely negated. In addition, the seeds of some species (e.g., cyclamen) may germinate fairly uniformly, but then may lose uniformity during subsequent developmental phases. In this case as well, the advantages offered by pregerminated seeds largely disappear. Moreover, many investigators have observed that storage life of such seeds is generally of limited duration or that specialized storage facilities are required.

U.S. Pat. No. 4,905,411 discloses pregerminated seeds having emerged radicles and a moisture content at which radicle development is suspended without the loss of seed viability. The pregerminated seeds described in this patent are prepared by germinating the seeds to a stage in which radicles have emerged, selecting those seeds having emerged radicles, and drying the seeds under conditions and to a moisture content which suspends radicle development but does not result in a loss of seed viability.

U.S. Pat. No. 5,573,827 discloses that a hydrogel may be applied to the pregerminated seed in order to improve plant growth by controlling the amount of cross-linking. U.S. Pat. Nos. 5,522,907 and No. 5,585,536 disclose pregerminated seeds that have desiccation-tolerant emerged radicles. According to these patents, the emerged radicle can be of any length up to the maximum diameter of a seed. These patents further illustrate that desiccation tolerance can be induced in seeds having an emerged radicle. Furthermore, it was found that seeds comprising desiccation-tolerant emerged radicles are capable of being sown without the need for employing refinements to sowing methods such as the application of encapsulating gels to pregerminated seed and the like. Despite these apparent advantages over raw or primed seed, the opportunity for creating a more developmentally-advanced, seed-derived form has remained available, especially for the reasons described immediately above.

There is a need in the art for a more developmentally-advanced seed- or somatic embryo-derived form that is capable of being stored for a period of time, and that is also able to be sown using conventional seed-sowing equipment. The latter requirement absolutely necessitated that a method be developed that allowed for the manipulation of the emerging radicle and/or hypocotyl beyond the maximum diameter of the seed. Of utmost importance is the ability to predictably and reliably control elongation of the root of the developed seed.

It is well known in the art that control of root development can be affected through various avenues of approach. Numerous investigators have identified the critical importance that calcium (Ca²⁺) ions play in the process of root development. The dual roles for calcium includes its structural role as an integral component of the cell membrane and its crucial role as a secondary messenger in signal transduction pathways that operate throughout the cell. The participation of calcium ions in these activities can be identified through the use of chemical reagents that chelate free calcium ions or other reagents, termed channel blockers, which in turn prohibit the uptake and utilization of calcium by the cell. Still other investigators have identified the importance of phytohormones in the development of primary and secondary roots. Among the classes of known phytohormones, the auxins are well known for their role in promoting the initiation of roots, both in vitro and in planta. Still other reports have identified the sensitivity of root development to osmotic compounds such as salt, polyethylene glycol (hereinafter referred to as “PEG”) and sugar alcohols like mannitol and sorbitol. Finally, others have demonstrated that an incomplete nutrient supply can significantly influence root development. One example includes the observation that ammonium ions can inhibit root development when potassium ions are absent (see Cao et al., Plant Physiol. 102:983-989 (1993)). Normal root development can be restored by the addition of potassium ions or other ions that closely resemble potassium (rubidium). Other lesser-known methods for affecting root development include exposure to heavy metals like copper and lead, herbicides, pH extremes, organic solutes, temperature extremes, high concentrations of ethylene, and compacted soils.

### BRIEF SUMMARY OF THE INVENTION

Despite this plethora of methods available to affect root development, the art does not teach whether root elongation can be restored after interrupting or inhibiting root development. According to the present invention, not only can root development be re-initiated and resumed, but root structures can be significantly enhanced by appropriate treatments. Furthermore, the ability to carefully manipulate root formation provides an opportunity to permit much more extensive development of the hypocotyl and cotyledon portions of the germinating seed. These developed seed forms possess an advanced developmental state as compared to raw, primed seed (imbibed, but no radicle protrusion), and pregerminated seed (as defined by U.S. Pat. Nos. 5,522,907 and No. 5,585,536) that exhibit protruded radicles. This level of control over the seed germination process and seedling development has led to the production of the developed seed forms of the present invention that are capable of being stored for a period of time and then sown using conventional seeding equipment.

The developed seed of the present invention can exist in either of two forms, the precotyledon form or the cotyledon form, as well as in a desiccation-tolerant form and desiccation-intolerant form. The precotyledon form has a modified root structure, which is characterized by a truncated root. The modified root structure may or may not be accompanied by a radial swelling which is located either at the base of the hypocotyl or distal to the hypocotyl. The cotyledonary leaves of the precotyledon form can remain enwrapped by an attached seed coat. The cotyledon form is a latter stage developmental form having a modified root structure comprising truncated roots. For those plant species characterized as having seed coats, the cotyledons are liberated from the seed coat and are exposed and are intensely green in color.

The most dramatic aspect of these two forms of developed seed is that the normal development of the root systems (e.g., root elongation) has been interrupted and altered. This feature ensures that the normally-long and entangled roots are absent and cannot interfere with the free-flowing and singulated nature of the developed seed. This result ensures that this product can be sown on commercially-available seeding equipment.

Generally, the present invention relates to developed seed and methods for making such developed seed. The developed seed of the present invention can be from, but is not limited to, the following plants: Alliums, Antirrhinums, Asteraceae, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Violas, Apiums, Daucuses, Chicoriums or Zinnias.

In one aspect, the present invention relates to a developed seed that contains a modified root structure and, optionally, an emerged hypocotyl. The developed seed can have its residual external moisture removed so as not to cause agglomeration, and is singulated and free-flowing and can be operationally sown in the same manner as raw, primed or pregerminated seed. The developed seed contains higher levels of chaperonin 60 when compared to raw, primed or pregerminated seed from the same plant species used as starting material for the developed seed.

The developed seed further exhibits enhanced rooting and earlier photosynthetic activity when compared to raw, primed or pregerminated seed from the same plant species. Additionally, the developed seed can further contain a seed coating.

In another aspect, the present invention also relates to plants grown from said developed seed. Such plants have a shorter internode length than a plant grown from raw, primed or pregerminated seed from the same plant species used as starting material for the developed seed.

In yet another aspect, the present invention relates to developed seed form that contains a modified root structure and an emerged hypocotyl. Like the developed seed described above, this form can have its residual external moisture removed so as not to cause agglomeration, and is singulated and free-flowing and can be operationally sown in the same manner as raw, primed or pregerminated seed. The modified root structure of the developed seed is characterized by a truncated appearance and has either a radial swelling at the base or distal to the hypocotyl and in some cases, secondary roots. Additionally, this developed seed can exhibit an exposed cotyledon(s) and the hypocotyl can display visibly green chlorophyll. Moreover, the developed seed contains higher levels of chaperonin 60 when compared to raw, primed or pregerminated seed from the same plant species used as starting material for the developed seed.

The developed seed described in the preceding paragraph above further exhibits enhanced rooting and earlier photosynthetic activity when compared to raw, primed or pregerminated seed from the same plant species. Additionally, the developed seed can further contain a seed coating.

In another aspect, the present invention also relates to plants grown from said developed seed. Such plants can exhibit a shorter internode length than a plant grown from raw, primed or pregerminated seed from the same plant species used as starting material for the developed seed.

An above-described developed seed can be present as a desiccation-tolerant or desiccation-intolerant form. As prepared, the above-described developed seed is desiccation-intolerant, but can be converted into desiccation-tolerant developed seed using well-known techniques. Contemplated desiccation-tolerant developed seed typically has a moisture content of about 4 to about 30 percent, and more usually about 20 to about 30 percent for seeds that do not exhibit seminal root systems and about 4 to about 12 percent for seeds that exhibit a seminal root system. A non-germinated seed typically has a moisture content of about 4 to about 12 weight percent.

In yet another aspect, the present invention relates to a method (process) for making a developmentally-advanced developed seed. The normal root development of the developed seed has been interrupted and altered. However, root development is not terminated, but instead is capable of resuming when the developed seed is sown in a suitable environment.

The first step of the method involves placing a batch of seed(s) or somatic embryos into a germination environment. The germination environment contains water and at least one auxin. The germination environment can also contain nutrients and/or at least one organic acid. In addition, the germination environment can also contain root-promoting compounds, calcium chelators, calcium channel blockers and light energy. The seed or somatic embryos is maintained in the germination environment a time period sufficient to form a precotyledon form of the developed seed. That time period for germination in the germination environment is typically about 0 days to about 50 days and at a temperature of from about 5° C. to about 30° C.

A contemplated process can also include further steps of separating a developed seed fraction from seed or somatic embryos that had not formed developed seed, and collecting a purified developed seed fraction. In addition, the seed or somatic embryos that had not formed developed seed can be maintained in said germination environment until a precotyledon form of the developed seed is obtained.

An optional step contemplates returning the remaining portions of the batch of seeds or somatic embryos after fractionation (i.e., those seeds separated away from the developed seed) to the germination environment for additional treatment times until the precotyledon or cotyledon forms are achieved, and then harvested again as described above. This step can be repeated numerous times to maximize the yield of the developed seed. The separated seed is typically collected and moisture is removed from the surface of the collected developed seed to form externally dried developed seed. The externally dried developed seed is slowly cooled and acclimated to refrigerated temperatures. Preferably, the developed seed is cooled over a period of from about 6 to about 20 hours to a temperature of from about 1° C. to about 15° C. Once cooled, the developed seed can be stored at a temperature of from about 1° C. to about 15° C.

In yet another aspect, the present invention relates to developed seed produced by the hereinbefore described methods and plants grown from said developed seed.

In a still further aspect, the present invention relates to a positive correlation between the advanced developmental stages achieved in the precotyledon and cotyledon forms of developed seed and the increased accumulation of a developmentally-regulated protein. Specifically, elevated levels of the organellar chaperonin, Cpn60, have been found to occur in the precotyledon and cotyledon forms of developed seed. Moreover, the increase in developed seed Cpn60 levels follows the normal, developmentally-regulated expression pattern observed in water-germinated seeds of the same plant species. Most importantly, the levels of Cpn60 are higher in developed seeds than in primed and pregerminated seeds of the same plant species (where they were measured to be at the same basal levels as raw seed), an observation that is entirely consistent with the advanced developmental state achieved in developed seeds.

### BRIEF DESCRIPTION OF THE FIGURES

The file of this patent contains at least one drawing executed in color. Copies of this patent with color drawings(s) will be provided by the Patent and Trademark Office upon request and payment of the necessary fee.

FIG. 1 shows the precotyledon (A) and cotyledon (C) forms of begonia developed seed. Begonia seeds germinated in water for the same period of time as the precotyledon and cotyledon forms are shown in (B) and (D), respectively. The truncated root, shortened hypocotyl and modest basal swelling in the precotyledon (A) compared to the water-treated seed (B) are visible. The truncated root, shortened hypocotyl and exposed cotyledons in the cotyledon form of developed begonia seed (C) compared to the water-treated seeds (D) are visible.

FIG. 2 shows the precotyledon (A) and cotyledon (C) forms of impatiens developed seed. The water-treated seed controls for the precotyledon and cotyledon forms are shown in (B) and (D), respectively. With respect to the precotyledon form (A), note the pronounced radial swelling at the base of the hypocotyl which is clearly absent in the water-treated seeds (B). For the cotyledon form (C), the truncated root, reduced hypocotyl length, fully exposed cotyledons and radial swelling at the base of the hypocotyl are features absent from the water-treated seeds (with the exception of the exposed cotyledonary leaves).

FIG. 3 shows the precotyledon (A) and cotyledon (C) forms of lisianthus developed seed. Lisianthus seeds germinated in water for the same period of time as the precotyledon and cotyledon forms are shown in (B) and (D), respectively. Basal radial swelling and the visibly green hypocotyl in the precotyledon form (A) are visible. In addition, the truncated root and shortened hypocotyl in the cotyledon form (C), features that are notably lacking in the water-treated seed (D), are visible.

FIG. 4 shows the precotyledon (A) and cotyledon (C) forms of pansy developed seed. Pansy seeds germinated in water for the same period of time as the precotyledon and cotyledon forms are shown in (B) and (D), respectively. For the pansy precotyledon (A), the dramatically truncated root and basal radial swelling which are totally lacking in the water-treated seeds (B), are visible. The truncated root of the cotyledon form (C) that is not found in the water-treated seed (D), is visible. All pansy seeds were previously primed.

FIG. 5 shows the precotyledon (A) and cotyledon (C) forms of pansy developed seed using pregerminated seed as the starting material. Pregerminated pansy seeds germinated in water for the same period of time as the precotyledon and cotyledon forms are shown in (B) and (D), respectively. The pregerminated pansy seeds can be induced to yield the same precotyledon (A) and cotyledon (C) forms as were produced with the primed pansy seeds (See FIG. 4).

FIG. 6 shows the precotyledon (A) and cotyledon (C) forms of petunia developed seed. Petunia seeds germinated in water for the same period of time as the precotyledon and cotyledon forms are shown in (B) and (D), respectively. The dramatically reduced hypocotyl length, basal radial swelling and visibly green hypocotyl in the precotyledon form (A), are visible. In addition, the truncated root and shortened hypocotyl in the cotyledon form (C) are visible.

FIG. 7 shows the precotyledon (A) and cotyledon (C) forms of salvia developed seed. Salvia seeds germinated in water for the same period of time as the precotyledon and cotyledon forms are shown in (B) and (D), respectively. Regarding the precotyledon form (A), basal radial swelling, truncated root and shortened hypocotyl that are not found in the water-treated seed (B), are visible. In the cotyledon form (C), the basal radial swelling, truncated root and the reduced hypocotyl length remain features which are absent in the water-treated seeds.

FIG. 8 shows the precotyledon (A) and cotyledon (C) forms of stock developed seed. Stock seeds germinated in water for the same period of time as the precotyledon and cotyledon forms are shown in (B) and (D), respectively. The truncated root and shortened hypocotyl in the precotyledon from (A) compared to the water-treated seed (B) is visible. In the cotyledon form (C), the truncated root, basal radial swelling and shortened hypocotyl, all features absent in the water-treated seeds, are visible. FIG. 9 shows the precotyledon (A) form of verbena developed seed. Verbena seed germinated in water for the same period of time as the precotyledon form is shown in (B). The precotyledon is characterized by the truncated root and basal radial swelling.

FIG. 10 shows the precotyledon (A) and cotyledon (C) forms of vinca (Catharanthus roseus) developed seed. Vinca seeds germinated in water for the same period of time as the precotyledon and cotyledon forms are shown in (B) and (D), respectively. The dramatically truncated root and basal radial swelling in the precotyledon form (A) are visible, and are completely absent in the water-treated seed (B). The precotyledon form (C) retains the basal radial swelling and the truncated root that are not found in the water-treated seed (D).

FIG. 11 shows the precotyledon (A) and cotyledon (C) forms of broccoli developed seed. Broccoli seeds germinated in water for the same period of time as the precotyledon and cotyledon forms are shown in (B) and (D), respectively. The precotyledon form exhibits a truncated root, unlike the water-treated seed (B). The cotyledon form (C) also exhibits a dramatically truncated root, along with a shortened hypocotyl, compared to the water-treated seed (D).

FIG. 12 shows the precotyledon (A) and cotyledon (C) forms of carrot developed seed. Carrot seeds germinated in water for the same period of time as the precotyledon and cotyledon forms are shown in (B) and (D), respectively. The precotyledon (A) displays the basal radial swelling and a shortened hypocotyl. These features are retained in the cotyledon form (C) as well (along with a truncated root).

FIG. 13 shows the precotyledon (A) and cotyledon (C) forms of cauliflower developed seed. Cauliflower seeds germinated in water for the same period of time as the precotyledon and cotyledon forms are shown in (B) and (D), respectively. The truncated root and shortened hypocotyl are the most prominent features of the precotyledon form (A). These features, along with the basal radial swelling, are the predominant features of the cotyledon form (C).

FIG. 14 shows the precotyledon (A) and cotyledon (C) forms of cucumber developed seed. Cucumber seeds germinated in water for the same period of time as the precotyledon and cotyledon forms are shown in (B) and (D), respectively. The precotyledon (A) and cotyledon (C) forms both display a truncated root.

FIG. 15 shows the precotyledon (A) and cotyledon (C) forms of lettuce developed seed. Lettuce seeds germinated in water for the same period of time as the precotyledon and cotyledon forms are shown in (B) and (D), respectively. The lettuce precotyledons (A) exhibit a truncated root, shortened hypocotyl and basal radial swelling. All of these readily-visible features are retained in the cotyledon form (C).

FIG. 16 shows the precotyledon (A) and cotyledon (C) forms of onion developed seed. Onion seeds germinated in water for the same period of time as the precotyledon and cotyledon forms are shown in (B) and (D), respectively. The severely truncated root structure in the precotyledon form (A) compared to the water-treated seed (B) is visible. The cotyledon forms (C) (although shown here with seed coats still attached since onion seed coats remain attached to the cotyledonary leaves for an exceptionally long period of time) exhibit the truncated, swollen root which is not seen in the water-treated seeds (D).

FIG. 17 shows the precotyledon (A) and cotyledon (C) forms of pepper developed seed. Pepper seeds germinated in water for the same period of time as the precotyledon and cotyledon forms are shown in (B) and (D), respectively. The truncated root and basal radial swelling in the precotyledon form (A) are visible. In the cotyledon form, the shortened hypocotyl and truncated, swollen root (C) which are absent in the water-treated seeds (D), are visible.

FIGS. 18 and 19 show the precotyledon (A) and cotyledon (C) forms of tomato developed seed (two varieties of tomatoes shown, specifically, Tumbler (FIG. 18) and Beefmaster (FIG. 19)). Tomato seeds germinated in water for the same period of time as the precotyledon and cotyledon forms are shown in (B) and (D), respectively. The precotyledons of both varieties (A) display a truncated root, shortened hypocotyl and marked basal radial swelling. The secondary roots that have been induced on the precotyledons of both varieties (see arrows), are visible. The cotyledon form displays a swollen, truncated root and shortened hypocotyl (C).

FIG. 20 shows the precotyledon (A) and cotyledon (C) forms of watermelon developed seed. Watermelon seeds germinated in water for the same period of time as the precotyledon and cotyledon forms are shown in (B) and (D), respectively. A truncated root and shortened hypocotyl in the precotyledon (A) and cotyledon forms (C) can be readily observed.

FIG. 21 shows the precotyledon form of cyclamen somatic embryos. Cyclamen somatic embryos were germinated either in germination medium (B) or in a developed seed-like solution (A) designed to control root development. Root development is completely inhibited in the precotyledon form.

FIG. 22 shows the cotyledon (A) form of Kentucky Bluegrass developed seed. Kentucky bluegrass seeds germinated in water for the same period of time as the cotyledon form are shown in (B). The water-treated seeds display a long primary root that is not detectable in the cotyledon form (A).

FIG. 23 shows the cotyledon (A) form of rice developed seed. Rice seeds germinated in water for the same period of time as the cotyledon form are shown in (B). The cotyledon form is characterized by a dramatically truncated root while the water-treated seeds display a long primary root.

FIG. 24 shows the precotyledon form of cyclamen developed seed.

### DETAILED DESCRIPTION OF THE INVENTION

Introduction

Normal root development in seeds takes place as follows: viable seeds start to germinate when provided with appropriate conditions of moisture, temperature, oxygen, and in some cases, light. The seeds imbibe water, the tissues swell, and the seed coat becomes soft and elastic. The radicle then pierces the seed coat. The radicle, the main root initial of the embryo, develops into the primary root after emergence through the seed coat during germination. The primary root is commonly white and slender and elongates rapidly. Numerous root hairs (fine protuberances of the outermost root cells) are usually abundant from the earliest developmental stages. Secondary roots are produced, either as lateral roots emerging from the primary root itself, or as adventitious roots emerging from the other parts of the seedling (e.g., hypocotyl). Development of the shoot system follows. The main functions of the root system are to anchor the plant in the soil, to absorb water and dissolved salts, and to conduct the water and salts to the cotyledons and the shoot. Roots may also be specialized for storage of food reserves.

In one embodiment, the present invention relates to a novel, developmentally-advanced, developed seed. The developed seed of the present invention can be derived from somatic embryos or seed, such as, but not limited to raw, primed or pregerminated seed. Specifically, suitable seed types that can be used in the present invention include those that are capable of forming root primordia from at least a hypocotyl region. Preferably, the developed seed of the present invention can be from any plant species, including, but not limited to, Alliums, Antirrhinums, Asteraceae, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Violas, Apiums, Daucuses, Chicoriums, and Zinnias. Also encompassed within the scope of the present invention are plants grown from the developed seed of the present invention.

The developed seed of the present invention is unique in that the root development of the developed seed has been interrupted and altered. However, even though the normal root development of the developed seed of the present invention has been interrupted, altered and modified, root development may be re-initiated and resumed when the developed seed is sown in a suitable environment as described herein. After the developed seed is sown in a suitable environment, a usable plant can be obtained. In fact, as will be shown in greater detail herein, the altered root development of the developed seed of the present invention permits for continued extensive development of the hypocotyl and cotyledon(s) during the formation of the developed seed.

In a further embodiment, developed seed can be present in a desiccation-tolerant or desiccation-intolerant form. As initially prepared and discussed in Examples 1-13 hereinafter, developed seed is desiccation-intolerant, but can be converted into desiccation-tolerant developed seed using well-known techniques, as is illustrated in Example 15. Contemplated desiccation-tolerant developed seed typically has a moisture content of about 4 to about 30 percent, and more usually about 20 to about 30 percent for seeds that do not exhibit seminal root systems and about 4 to about 12 percent for seeds that exhibit a seminal root system.

In another embodiment, the present invention relates to methods for making such a developmentally-advanced, developed seed having its root development interrupted and altered.

In another embodiment, the present invention relates to a method of manipulating the growth habit of young plants that are derived from the developed seed of the present invention. Specifically, the young plants derived from the developed seed of the present invention can exhibit a dramatically-reduced stature or compact phenotype, are highly toned and require fewer applications of plant growth regulators to control excessive growth.

In a further embodiment, the present invention relates to a method for improving the quality of seed lots with respect to increasing the percentage of usable young plants obtained from such seed lots.

Definitions

The headings provided herein are not limitations of the various aspects or embodiments of the invention, which can be had by reference to the specification as a whole. Accordingly, the terms defined immediately below are more fully defined by reference to the specification as a whole.

As used herein, the term “hypocotyl” means the axial part of an embryo or seedling located between the cotyledon or cotyledons and the radicle.

As used herein, the term, “cotyledon(s)” means one or more leaf-like appendages that develop from embryos of seed plants.

As used herein, the term, “radicle”, means the embryonic root that forms the basal continuation of the hypocotyl in an embryo.

As used herein, the term “primoridum” or “primoridia” means an organ, a cell, or an organized series of cells in their earliest stage of differentiation (e.g., leaf primordium).

As used herein, the term “raw seed” means seed that has not been treated; specifically, seed that has not been primed, pregerminated or pelleted.

As used herein, the term, “pregerminated seed” means seeds undergoing the biochemical and physiological processes of seed germination up to the point of radicle protrusion.

As used herein, the term, “primed seed”, means seeds which have been soaked in an aerated, low water potential osmotica such as polyethylene glycol or salts, followed by subsequent drying in order to enhance germination, stand establishment and seedling growth.

As used herein, the term “primary root” means the root developing in continuation of the radicle of an embryo.

As used herein, the term “secondary root” means any root other than the primary root and includes lateral root and adventitious root.

As used herein, the term “lateral root” means a root arising from another root.

As used herein, the term “adventitious root” means roots arising not from their usual sites, as roots originating on stems instead of on other roots.

As used herein, the term “seed coat” means the outer covering of a seed derived from the integument(s).

As used herein, the term “somatic embryo” means an embryo developing not from the direct product of gametic fusion.

As used herein, the term “zygotic embryo” means a young sporophyte of a seed plant.

As used herein, the term “modified root structure” means a truncated root that may or may not be accompanied by secondary roots and/or radial swelling at the base of or distal to the hypocotyl.

As used herein, the term “germination” means a physiological process that begins with water uptake and ends with the start of elongation by the embryonic axis, usually the radicle.

As used herein, the term “pericarp” means the ovary wall. The pericarp can be thin and fused with the seed coat as in corn, fleshy as in berries, or hard and dry as in pods of legumes.

As used herein, the term “developed seed” means any plant propagate that contains embryonic tissue which, under the appropriate conditions, that results in the growth and development of a plant body. These include zygotic embryos, parthenogenic seeds, somatic embryos, and other plant propagules such as potato seed pieces, beet seeds (fruits), cereal seeds (caryopses), etc., which will result in plant growth. Developed seed exists in two (2) forms: (1) the precotyledon form; and (2) cotyledon form.

As used herein, the term “precotyledon form” means a developed seed characterized by a modified root structure, an attached seed coat, and optionally, an emerged hypocotyl.

As used herein, the term “cotyledon form” means a developed seed characterized by a modified root structure, an emerged hypocotyl and an exposed cotyledon(s).

As used herein, the term “toning” or “toned” refers to the slowing of growth and thickening of the leaves and stems of a seedling or young plant which allows a seedling or young plant to withstand holding, shipping or harsh transplanting conditions.

As used herein, the term “moisture content” refers to the moisture content of a developed seed calculated on a fresh weight basis. Rules for determining moisture content as defined herein have been promulgated by the International Seed Testing Association in Seed Science and Technology, 4:40-43 (1976).

As used herein, the term “desiccation-tolerant” developed seed means developed seed whose viability does not decrease with a moisture content between about 12 and about 31 percent.

As used herein, the term “desiccation-intolerant” or “desiccation sensitive” or “recalcitrant” developed seed mean developed seed whose viability decreases with a moisture content between about 12 and about 31 percent.

As used herein, the term “unusable botanic seed” refers to seed or somatic embryos that do not yield a normal seedling.

As used herein, the term “singulated” means being of an individual form.

Developed Seed Product

In one embodiment, the present invention relates to developed seed wherein the normal root development of the developed seed has been significantly, but not irreversibly interrupted, and altered. Several molecular mechanisms may act simultaneously to alter the normal root development of developed seed and can instead re-direct preferred development to the hypocotyl and cotyledon(s) of the developed seed.

As described previously, although normal root development in the developed seed of the present invention has been interrupted and altered without significantly impacting hypocotyl and cotyledon(s) growth, root development is not terminated. Rather, root development is re-initiated and resumed once the developed seed is sown in a suitable environment. As used herein, the term “suitable environment” means conditions of temperature, oxygen, moisture, light and nutrients that are appropriate for continued plant growth and development. In fact, once the developed seed of the present invention is sown in a suitable environment, root initiation and elongation not only proceed, but also proceed in an expeditious manner. Furthermore, the inventors have found that the modified root formation permits extensive continued development of the hypocotyl and cotyledon portions of the developed seed during the developed seed process.

It is known in the art that root growth in Arabidopsis thaliana can be inhibited by germinating Arabidopsis seeds in a medium lacking potassium ions (see Cao et al., Plant Physiol., 102:983-989 (1993)). It is also known that such inhibition of root growth can be reversed only by adding such potassium ions back to the growth medium. However, Cao et al. do not teach that root development can be restored or re-initiated after extended periods of time in storage. Nonetheless, as discussed earlier, with respect to the developed seeds of the present invention, root development is re-initiated and resumed once the developed seed is sown in a suitable environment without the addition of any nutrients, minerals or chemicals, such as potassium. Moreover, with respect to the developed seeds of the present invention, root development can be restored or-initiated after extended periods of time in storage.

Moreover, the inventors of the present invention have discovered that the experimental conditions reported by Cao et al. to inhibit root development are extremely limited in their applicability (described in U.S. Application No. 60/148,354, herein incorporated by reference). For example, the inventors discovered that inhibition of root growth in germinating Arabidopsis seeds was possible only by using solidified, ammonium ion-containing, potassium ion-deficient nutrient medium (as reported by Cao et al.). When the seeds were germinated in a liquid environment of the same nutrient medium (containing ammonium, but lacking potassium), no root inhibition was observed. It was observed that root development proceeded normally and irrespective of the ionic conditions in the liquid nutrient medium. Thus, the inhibitory effect on root development was strictly environment-dependent.

As a second example of the limitations of Cao et al., the inventors found that root inhibition in germinating impatiens seeds was not impacted by the ionic conditions of the nutrient medium, but rather, by osmotic conditions (described in U.S. Application No. 60/148,354, herein incorporated by reference). The inventors learned that root development in germinating impatiens seeds was unaffected by an ammonium/potassium ionic imbalance, but was severely inhibited by the inclusion of 1% (w/v) sucrose in the nutrient medium (the same concentration reported by Cao et al.). It was discovered that the sucrose, in either a liquid or solid environment, dramatically inhibited impatiens root development. This result stands in sharp contrast to the results obtained with Arabidopsis seeds where addition of 1% (w/v) sucrose to the nutrient medium was observed by the inventors to actually stimulate root development. Also different, Arabidopsis, but not impatiens, root development was sensitive to an ammonium/potassium ionic imbalance in the nutrient medium.

As a further example, the inventors learned that the root-inhibiting treatments of Cao et al. do not guarantee that a usable plant can be recovered (described in U.S. Application No. 60/148,354, herein incorporated by reference). It was learned that at least one of the root-inhibiting treatments described by Cao et al., when applied to germinating impatiens seeds, led to an irreversible cessation in seedling growth and development. Taken altogether, these results demonstrate that the methods of root inhibition described by Cao et al. ultimately teach only how to inhibit root development in germinating Arabidopsis seeds on a solid (specifically) nutrient medium containing an ammonium/potassium ionic imbalance. The inventors found that these experimental conditions cannot be assumed to generally apply to other germinating seeds (e.g., impatiens) to achieve the same results.

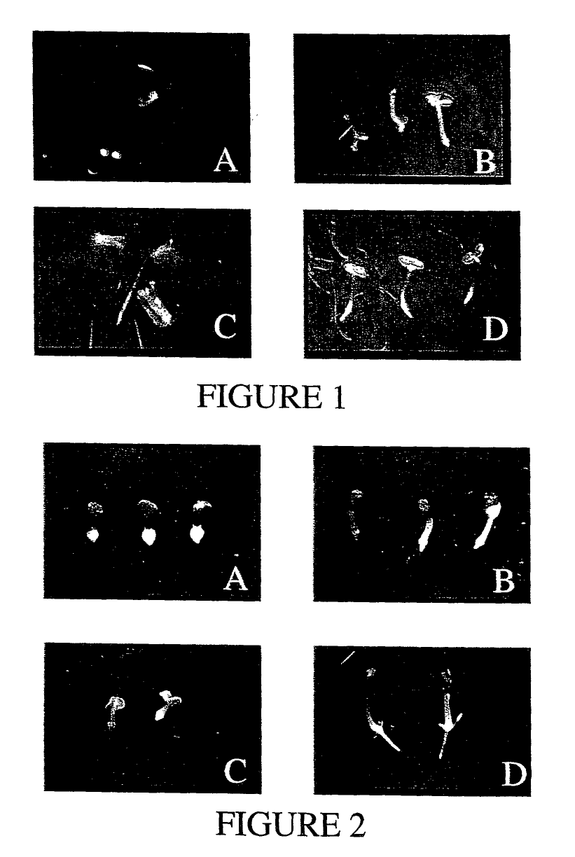

As a result of the normal root development of the developed seed of the present invention being interrupted and altered, the developed seed can develop one or more of the following clearly visible and uniquely identifiable features: (1) a modified root structure; and optionally, (2) an emerged hypocotyl; and optionally, (3) exposed cotyledon(s). The modified root structure of the developed seed of the present invention exhibits a truncated appearance when compared to water-germinated seed of same variety. It is known in the art that roots can be truncated physically by cutting roots with appropriate root-cutting instruments. However, the developed seed of the present invention grows and develops a naturally-truncated root. As shown in FIGS. 1-23, the developed seed of the present invention exhibits a significantly shorter root when compared with water-treated seed controls of the same variety. Moreover, in addition to displaying a truncated appearance, the modified root structure of the developed seed of the present invention can also exhibit at least one of the following: (1) swelling at the base of the hypocotyl or distal to the hypocotyl; or (2) a proliferation of secondary roots in certain plant species, such as impatiens and lisianthus. The developed seed of the present invention can exhibit a greater number of secondary roots when compared to water-germinated seeds from the same variety. Additionally, the secondary roots of the developed seed of the present invention can appear earlier in development when compared to water-germinated seeds of the same variety.

The modified root structure of the developed seed of the present invention is particularly advantageous for several reasons. Specifically, the modified root structure permits a product which can have its residual external moisture removed (for sowing purposes) and is singulated and free-flowing and therefore fully compatible with commercially available seed sowing methods. In fact, the developed seed of the present invention may be sown naked, if so desired (e.g., using conventional seed sowing methods and equipment without the need for employing encapsulating gels and the like). Additionally, because the modified root structure of the developed seed is extremely short, there is no entanglement of the root structure during the sowing process.

In addition to the modified root structure, the developed seed of the present invention can also contain an emerged hypocotyl. As shown in FIGS. 1-20 and FIGS. 22-23, the length of the emerged hypocotyl of the present invention is significantly shorter than the length of the hypocotyl from water-treated seed of the same variety (this is important to maintain ease of sowing). In addition, the hypocotyl of the developed seed of the present invention may exhibit visibly green chlorophyll that indicates that photosynthesis has been initiated in this tissue.

In another embodiment, the developed seed of the present invention can also possess a seed coat. The cotyledonary leaves of the developed seed can remain enwrapped by the seed coat. Alternatively, the cotyledons of the developed seed can be exposed and liberated from the seed coat (if a seed coat was originally present).

The developed seed of the present invention can exist in two (2) forms that are referred to herein as the “precotyledon form” and the “cotyledon form”. An obvious feature of the precotyledon form is its modified root structure, which is characterized by a truncated root. The modified root structure may or may not be accompanied by radial swelling which is located either at the base of the hypocotyl or distal to the hypocotyl. In the plant species tested, a shortened hypocotyl is typical of the precotyledon form. Additionally, the shortened hypocotyl of the precotyledon form can be distinctly green due to the photosynthetic processes that have initiated and are ongoing in this tissue. Finally, the cotyledonary leaves of the precotyledon form can remain enwrapped by an attached seed coat.

The cotyledon form is a latter stage developmental form having a modified root structure possessing truncated roots. In the cotyledon form, for those species that have seed coats, the cotyledons are liberated from the seed coat and are exposed and are intensely green due to their already-established photosynthetic activity. While not wishing to be bound by any theory, it is believed that it is the organic acid employed in the germination environment during the process of making developed seed of the present invention which contributes to the cotyledons having such an intensely green color. More specifically, the inventors believe that the organic acid lowers the pH of the germination environment in the localized vicinity of the plasma membrane which in turn affects the ionic composition of the plasma membrane of the seed or somatic embryos used to make the developed seed of the present invention.

The developed seed of the present invention exhibits enhanced rooting when compared to raw, primed or pregerminated seed when sown in a suitable environment. Enhanced rooting can be determined by measuring the root area (in mm²) of developed seed and raw, primed or pregerminated seed. Root area can be measured using suitable techniques known in the art. For example, seedlings can be photographed with a CCD camera and the total root area calculated using Quantimet Image Processing Software (hereinafter “QUIPS”) as described in U.S. Pat. Nos. 5,659,623 and No. 5,572,827, herein incorporated by reference. The developed seed of the present invention has been found to exhibit enhanced rooting when compared to raw seed one, two or three (1, 2 or 3) days after the developed seed and raw seed are sown in a suitable environment.

The developed seed of the present invention also demonstrates earlier photosynthetic development when compared to raw, primed or pregerminated seed when sown in a suitable environment. The photosynthetic development of developed seed and raw, primed or pregerminated seed can be determined by measuring photosynthetic activity, which can be determined using suitable techniques known in the art. For example, photosynthetic activity can be measured using a fluorometer. A fluorometer applies a pulse-modulated measuring light for selective detection of chlorophyll fluorescence yield, which is a measure of photosynthetic activity. The precotyledon form of the present invention exhibits earlier photosynthetic development when compared to raw seed at least one (1) day after sowing the precotyledon form and the raw seed in a suitable environment. The cotyledon form of the present invention exhibits earlier photosynthetic development when compared to raw seed upon sowing the cotyledon form and the raw seed in a suitable environment.

Pregerminated seeds having emerged radicles and a moisture content at which radicle development is suspended without a loss of seed viability are known in the art (see U.S. Pat. Nos. 4,905,411, No. 5,522,907 and No. 5,585,536). It is also known in the art that the emerged radicle can be of any length up to the maximum diameter of the seed (see U.S. Pat. Nos. 5,522,907 and No. 5,585,536). The developed seeds of the present invention are developmentally more advanced then the pregerminated seeds known in the art. As shown in Example 2, the pregerminated seeds known in the art can be used as the source or starting material to obtain the developed seeds of the present invention. Also as shown in Example 14, the developed seeds of the present invention contain higher levels of a germination-induced protein (Cpn60) than pregerminated seeds, another indication of their advanced developmental state.

Accumulation of Cpn60 in Developed Seed

In another embodiment, it has been discovered that the developed seed of the present invention contain higher levels of the chaperonin, Cpn60, than raw, primed or pregerminated seed.

Molecular chaperones are a class of essential proteins whose function is to ensure the correct folding and assembly of other polypeptides into oligomeric structures of which the chaperones are not a component. Chaperonins are a sub-class of chaperones, to which belong the family of heat-shock proteins with a molecular mass of 60,000 Da that include GroEL in Escherichia coli, ribulose 1,5-bisphosphate carboxylase oxygenase (Rubisco) subunit binding protein (RBP or plastid Cpn60) in chloroplasts, and mitochondrial Cpn60 in that organelle. The genes encoding chaperonins from several organisms have been cloned and the derived amino acid sequences show a very high degree of conservation from prokaryotes to eukaryotes. Some of the chaperonins are known to be heat-shock proteins in both prokaryotes and eukaryotes. However, plant Cpn60s are not generally considered to be heat-inducible proteins.

It is known that several chaperonins are developmentally-regulated in various plants and animals under normal conditions. Early events in seed germination have been used to define mechanisms by which mitochondrial biogenesis is regulated. Electron microscopic studies show that quiescent seed tissues contain poorly differentiated mitochondria but, upon imbibition, they become enlarged and develop complex inner membrane structures. The development of functional mitochondria during imbibition was reported to be dependent upon de novo protein synthesis in peanut cotyledons and on the activation of preformed mitochondria in pea cotyledons. It was also reported in germinating maize embryos that the cyanide-sensitive mitochondrial electron transport system was required for embryo germination and this activity depended upon newly synthesized or assembled respiratory enzyme complexes. Moreover, in germinating maize and Arabidopsis seeds, mitochondrial Cpn60 levels increased for the first 48 hours of seed germination. These observations taken together indicate an essential role for mitochondrial Cpn60 in the assembly of multi-subunit enzymatic complexes in developing mitochondria of germinating seeds.

Prior to its recognition as the chloroplast Cpn60, the large subunit binding protein was implicated in the assembly of the higher plant Rubisco, a hexadecamer comprised of eight large and eight small subunits. Assembly occurs in the chloroplast stroma, following post-translational import of the small subunits. However, numerous studies have shown that the holoenzyme does not assembly spontaneously. Indeed, the nascent large subunits initially form a stable complex with chloroplast Cpn60. Then, in a complicated and poorly understood set of reactions, the bound large subunits are discharged in an ATP-dependent manner and are subsequently incorporated into the Rubisco holoenzyme. More generally, a variety of proteins imported into isolated chloroplasts stably interact with chloroplast Cpn60. These findings suggest that chloroplast chaperonins play a prominent role in plastid protein folding. As per mitochondrial Cpn60, these observations suggest that chloroplast Cpn60 levels should also rise during the initial events of seed germination and seedling establishment, since this chaperonin would be required by the seedlings during the process of plastid development and differentiation into chloroplasts to gain photosynthetic competency.

The developed seed of the present invention exhibit elevated levels of Cpn60 after harvest from the germination environment when compared with raw, primed or pregerminated seeds of the same plant species used as starting material for the developed seed. For instance, as shown in Example 14, the precotyledon and cotyledon forms of developed seed from impatiens exhibit levels of Cpn60 of about forty-six percent (46%) (for the precotyledon form) and about one hundred and fifteen percent (115%) (for the cotyledon form) greater than the input primed impatiens seeds. Thus, such increases in the levels of Cpn60 in the developed seed provide a reliable and faithful indicator of the advanced developmental stages achieved in the precotyledon and cotyledon forms of the developed seed. The increase in Cpn60 content in the developed seed forms (namely, the precotyledon and cotyledon forms) relative to raw, primed or pregerminated seed of the same plant species used as the starting material provides a useful and measurable molecular marker to differentiate and distinguish developed seed from other seed enhancement techniques, such as, but not limited to, priming and pregermination.

Coated and Pelleted Developed Seed

In another embodiment, the present invention relates to coated developed seeds. As used herein, the term “coated developed seeds” refers to the description provided above for “developed seeds” except that the seeds are provided with an additional protective layer or in pelleted form. The pelleting material may comprise any conventional material commonly used in the art for protecting or pelleting seed. Suitable pelleting materials include clays such as sub-bentonite and bentonite, vermiculite along with additives such as perlite, pumice, metal stearates, polyethylene, polystyrene, polyurethane, talcum powder, polypropylene, polyvinyl chloride, starches, loams, sugars, arabic gums, organic polymers, celluloses, and flours such as wood flours, quartz powders and the like. Additionally, a hydrogel may be applied to the developed seed in order to improve plant growth by controlling the amount of cross-linking as described in U.S. Pat. No. 5,573,827, which is herein incorporated by reference.

These materials may be added to the developed seeds of the present invention using conventional layering or pelleting procedures that are well known in the seed technology arts. The pelleting material may also contain additional components that provide some advantage or benefit to the seed such as, but not limited to growth regulators, fungicides, insecticides and micronutrients.

The developed seed of the present invention can have its residual external moisture removed so as not to cause agglomeration and is singulated and free-flowing, and can be operationally sown in the same manner as raw, primed or pregerminated seed using techniques which are well-known in the art.

In another embodiment, the inventors have discovered that the developed seed of the present invention can be obtained from seeds that have been deemed to be commercially unusable. Unusable seed can be converted into the developed seed of the present invention and hence into a commercially usable product, using the methods described herein.

In another embodiment, the present invention relates to usable young plants or seedlings grown from the developed seed of the present invention. Usable plants have been obtained from the developed seed for every plant species shown in Example 1. After the developed seeds of the present invention are sown in a suitable environment, the young plants or seedlings resulting from said developed seeds can exhibit many beneficial attributes, such as a dramatically reduced stature or compact phenotype (due to reduced internode length), and are highly toned. Additionally, because these plants exhibit a compact phenotype, they require fewer applications of plant growth regulators to control excessive growth. The compact phenotype and highly-toned nature of the young plants or seedlings aids in shipping. Specifically, because the young plants or seedlings are small in stature, more young plants can be loaded into a truck for shipping. The advantage of the highly-toned nature of the young plants or seedlings is that it allows these young plants or seedlings to better withstand the numerous stresses and rigors of shipping. Finally, because these young plants or seedlings require fewer applications of plant growth regulators to control excessive growth, the grower is able to reduce costs (both labor and chemicals) in producing these plants.

Methods for Making Developed Seeds

In another embodiment, the present invention relates to a method (process) for making the developed seed of the present invention. The method of the present invention employs a novel germination environment that serves two (2) purposes. The first and primary purpose of this germination environment is to interrupt and alter the normal root development of a seed or somatic embryo. The second purpose of the germination environment is to nutritionally fortify the cotyledonary leaves and hypocotyl.

The developed seed of the present invention can be prepared as follows: seeds, such as, but not limited to, raw, primed or pregerminated seed or somatic embryos are placed into a suitable germination environment. As used herein, the term “germination environment” means an environment wherein seeds or somatic embryos may freely germinate at least to the extent that radicle protrusion occurs. The germination environment contains water and at least one auxin. Thus, the germination environment must be adequately moist, aerated or oxygenated, and capable of promoting germination to at least the stage of radicle protrusion from the seed coat or pericarp. One example of a suitable germination environment that can be used in the method of the present invention is an aerated water column. The aerated water column should have a degree of aeration that is sufficient to keep the seeds or somatic embryos of interest buoyed or in suspension. The amount of seed per unit volume can be any suitable amount, such as from about 1 gram to about 200 grams of seed per liter. Preferably, the amount of seed in the aerated water column is not more than about 25 grams of seeds per liter of water. However, one of ordinary skill in the art will recognize that the amount of seed per unit volume of water will be species-dependent.

Another suitable germination environment that can be used to prepare the developed seed of the present invention is moistened filter paper. The moistened filter paper may be placed on a tray or in a petri dish using any suitable technique. Another suitable germination environment that can be used to make the developed seed of the present invention is a moistened solid matrix, such as vermiculite, perlite or cellulose.

The temperature of the germination environment is one that permits or promotes the germination of seed or somatic embryos. The temperature of the germination environment will be species-dependent and can be experimentally determined. Generally, the temperature of the germination environment is from about 5° C. to about 30° C., depending on the species. Preferably, the temperature of the germination environment is from about 15° C. to about 25° C.

The germination environment contains a water-containing germination solution that contains at least one auxin used to produce the developed seed of the present invention. Preferably, in addition to at least one auxin, the germination solution also contains nutrients and/or at least one organic acid. Additionally, the germination solution may contain excipients, diluents, additives, factors, regulators and process enhancers as required, which may help in promoting or improving germination, maintaining primary root viability, or enhancing secondary root primordia induction in the developed seed.

The individual roles assumed by the components of the germination solution partially overlap and interact with one another in such a manner as to create in the germination environment, specifically, a nutrient imbalance and deficiency which interrupts and alters the normal root development of the seed or somatic embryos. While not wishing to be bound by this theory, the inventors believe that the individual roles assumed by the components of the germination solution partially overlap and interact with one another in such a manner so as to create in the germination environment conditions that are unfavorable for root development in germinating seeds. More specifically, the germination solution can be composed of multiple components that simultaneously exert multiple mechanisms that together can interrupt and alter root development. These mechanisms can include, but are not limited to: a) a nutrient imbalance in which a nutrient deficiency exists in at least one of the minerals calcium and magnesium; b) auxins which can affect calcium utilization and ultimately, root elongation; and c) an organic acid capable of chelating calcium and also affecting nutrient and ion uptake.

As discussed hereinbefore, the germination solution contains at least one auxin. Examples of auxins that can be used in the germination solution include, but are not limited to, indole-3-butyric acid (“IBA”), naphthaleneacetic acid (“NAA”), 2,4-dichlorophenoxyacetic acid, indole-3-acetic acid, indole-3-acetic acid methyl ester, indole-3-acetyl-L-alanine, indole-3-acetyl-L-aspartic acid, indole-3-acetyl-L-phenylalanine, indole-3-propionic acid, p-chlorophenoxyacetic acid, β-naphthoxyacetic acid, dicamba, picloram and combinations thereof.

The auxin is preferably present in the germination solution in the amount of about 0.005 ppm to about 500 ppm by volume of germination solution. The preferred auxin used in the germination solution of the present invention is IBA or NAA or combinations thereof. Preferably, IBA is present in the germination solution in the amount of about 0.1 to about 25 ppm by volume of germination solution and NAA is present in the germination solution in the amount of about 0.005 to about 50 ppm by volume of germination solution. The IBA is useful in the germination solution because it: (1) promotes root initiation; (2) inhibits root elongation; and (3) prevents calcium utilization. The NAA is useful in germination solution because it: (1) promotes root initiation; (2) inhibits root elongation; (3) prevents calcium utilization; and (4) promotes uniformity of seed response. It is believed that because the auxin used in the germination solution prevents calcium utilization, that the auxin plays a significant role in creating and maintaining the nutrient imbalance and deficiency which interrupts and alters the root development of the seed or somatic embryos.

In addition to at least one auxin, the germination solution can also contain nutrients and/or at least one organic acid. With respect to the nutrients, the amount and types of nutrients used in the germination solution are species-dependent. Any nutrients that promote the growth and development of the developed seed can be used in the germination solution. Preferably, the nutrients add ammonium and potassium ions to the germination solution.

Most preferably, the germination solution contains the following nutrients: fertilizer(s), vitamins, and potassium nitrate (KNO₃) and combinations thereof. Examples of fertilizers that can be used in the germination solution include Peters Fertilizer and Peters Stem Fertilizer (both available from Peters Professional® Fertilizer, The Scotts Company, 14111 Scottslawn Road, Marysville, Ohio). Peters Fertilizer and Peters Stem Fertilizer contain the following components: 0.2% ammonium nitrate, 0.2% potassium nitrate, 0.08% iron sulfate, 0.01% boric acid, 0.08% manganese sulfate, 0.04% zinc sulfate, 0.01% sodium molybdate, 0.03% copper sulfate, 0.1% ammonium phosphate and 0.15% sulfur. Peters Fertilizer and Peters Stem Fertilizer are collectively referred to herein as “Peters Fertilizer”). An example of another fertilizer that can be used in the present invention is Miracle-Gro®, which is also available from The Scotts Company. Examples of vitamins that can be used in the germination solution are AGRONOMIX® (a multi-vitamin mixture that contains one or more of the following: ascorbic acid, biotin, pyridoxine-HCl, thiamine hydrochloride, thiamine mononitrate, riboflavin, folic acid, niacinamide, pantothenic acid and inert carriers), which is commercially available from Vitech Enterprises, Inc, Palisades, N.J. 07054, and SUPERthrive®, which is commercially available from the Vitamin Institute, Box 230, North Hollywood, Calif. 91603.

The germination solution can preferably contain about 0.1 ppm to about 1000 ppm by volume of germination solution nutrients, preferably, about 250 ppm to about 350 ppm by volume of germination solution nutrients. Most preferably, the germination solution contains a fertilizer that is present in the amount of about 0.1 ppm to about 1000 ppm by volume of germination solution, preferably in the amount of about 100 ppm by volume of germination solution, vitamins, which are present in the amount of about 0.1 ppm to about 1000 ppm by volume of germination solution, preferably in the amount of about 100 ppm by volume of germination solution, and KNO₃ that is present in the amount of about 0.1 ppm to about 1000 ppm by volume of germination solution, preferably in the amount of about 100 ppm by volume of germination solution.

In addition to at least one auxin and nutrients, the germination solution can also contain at least one organic acid. As used herein, the term, “organic acid” includes carboxylic acids (contain —COOH groups) including aliphatic carboxylic acids (such as formic and acetic acids) and aromatic carboxylic acids (such as benzoic and salicylic acids), dicarboxylic acids (containing two —COOH groups), such as oxalic, phthalic, sebacic and adipic acids, fatty acids (contain —COOH group), including aliphatic fatty acids (such as oleic, palmitic and stearic fatty acids) and aromatic fatty acids (such as phenylstearic fatty acids). Preferred organic acids for use in the present invention, include, but are not limited to, citric acid, malic acid, maleic acid, malonic acid, ascorbic acid or combinations thereof. The organic acid is preferably present in the germination solution in the amount of about 0.01 mM to about 100 mM by volume of germination solution, preferably in the amount of 0.5 mM by volume of germination solution. It is believed that the organic acid minimizes hypocotyl elongation and facilitates nutrient uptake.

Another important component of the germination environment is light energy. Light energy is used to (1) inhibit hypocotyl elongation; and (2) stimulate photosynthesis. The seed or somatic embryos can be exposed to the light energy for a period of time of several minutes or several hours each day that they are in the germination environment. Or, the seed or somatic embryos can be exposed to the light energy continuously during their time in the germination environment. Preferably, the seed or somatic embryos are exposed to light energy for a period of at least about 16 hours a day while they are in the germination environment.

Optionally, the germination solution can also contain process enhancers. As used herein, the term “process enhancers” refers to any chemical or physical compounds or components that improve the overall efficiency of the developed seed method. Process enhancers that can be used in the germination solution of the present invention include, but are not limited to: root-promoting compounds such as dithiothreitol, cysteine, glutathione and β-mercaptoethanol, calcium chelators such as ethylene glycol-bis (β-aminoethyl ether)-N,N,N′,N′-tetraacetic acid (EGTA), which are compounds which bind free calcium ions; or other calcium channel blockers, such as lanthanum or manganese, which prohibit the uptake and utilization of calcium by the cell, and plant growth regulators, such as, but not limited to cytokinins (such as zeatin), gibberellins (such as GA₃), abscisic acid, ethylene and brassinosteroids.

The seeds or somatic embryos are maintained in the germination environment for a time period sufficient to form a precotyledon form to the developed seed. That time period is typically about 0 days to about 50 days, depending on the species. The term “0 days” refers to any length of time less than 24 hours.

Once the precotyledon or cotyledon form is obtained, the developed seed portion or fraction is separated from seed or somatic embryos that had not formed developed seed using suitable techniques known in the art. Preferably, once the desired developed seed form is obtained (e.g. precotyledon or cotyledon); it is removed from the germination environment without permitting any additional period of time for further growth. Typically, separation techniques rely on physical differences between germinated seed and non-germinated seed such as size, weight, shape and the life. One skilled in the art will recognize and appreciate the ease in which the developed seeds (shown in FIGS. 1-20 and 22-23) can be selected using any suitable technique. The separated developed seed is then collected.

It is well-known in the art that plant seeds, even those from the same seed lot of the same variety, which have been harvested and conditioned at the same time, do not germinate and develop synchronously after sowing. Specifically, the time and rates of germination are known to be influenced by many known and unknown determinants that are collectively referred to as “seed quality factors”. It has also been observed that the conversion of raw, primed or pregerminated seeds into the precotyledon and cotyledon forms of developed seed occur at different rates and extents for seeds from the identical seed lots. Just as germination and seedling development are non-synchronous under normal germination conditions, the generation of developed seed in the germination environment was found to be non-synchronous. Thereupon, it has been discovered that multiple, sequential harvests of the developed seed from the same germination environment can be used to achieve satisfactory yields (the term “yield” meaning the percentage of developed seeds harvested relative to the starting number of input seeds) which are commercially-acceptable.

More specifically, it has been discovered that once a number of seeds from a seed lot (such as raw, primed or pregerminated seeds) or somatic embryos contained in the germination environment, as described herein, have reached the desired developed seed form namely, the precotyledon or cotyledon form, the entire lot of seeds or embryos are harvested from the germination environment and the developed seeds separated from the seed or somatic embryos that had not formed developed seed (non-developed seed forms). The seeds can be separated using any technique known in the art, such as buoyant density separation. The separated non-developed seed forms are then returned to the germination environment and maintained there for further treatment. The seeds can be removed daily or every few days and the developed seeds separated from the seed or somatic embryos that had not formed developed seed using the same techniques as described above. The seed or somatic embryos that had not formed developed seed are then returned to the germination environment. This process is continued until greater than fifty percent (50%), preferably greater than seventy-five percent (75%), and most preferably, greater than eighty-five percent (85%) of the initial seed lot reach the desired developed seed form.

Once the developed seed is removed from the germination environment, it is preferably handled to remove residual external moisture, using techniques known in the art. Preferably, the developed seed is dried to a relative moisture content of from about fifty percent (50%) to about ninety-five percent (95%) using dewatering techniques well known in the art, such as, but not limited to a vacuum, active drying, and the like.

Once the developed seed has had residual external moisture removed, it can be stored under suitable storage conditions. For example, the developed seed of the present invention can be stored at refrigerated temperatures between about 1° C. to about 15° C. Preferably, once the external moisture has been removed from the developed seed, it is cooled over a period of about 6 to about 20 hours to a temperature of from about zero ° C. to about 15° C. Most preferably, the developed seed is cooled over a period of about 18 hours to a temperature of about 5° C. and then stored at a temperature of 5° C.

Developed seed contemplated herein is not tolerant to desiccation as it is prepared. That is, the developed seed cannot be dried to a moisture content of about 4 to about 30 percent and maintain its typically 85 to 100 percent ability to grow into usable plants after being planted in a suitable medium. This is particularly the case for plants that exhibit seminal root growth.

Thus, as is shown in Example 15, impatiens that exhibit seminal root growth could be dried to a moisture content of about 4 to about 12 percent and still exhibit about 45 to about 65 percent growth as compared to 100 percent growth for the as formed developed seed. Similarly dried vinca developed seed that do not exhibit seminal root growth exhibited zero percent growth after drying.

On the other hand, when those same impatiens developed seeds were treated with PEG-8000 as discussed in U.S. Pat. No. 5,522,907 to form desiccation-tolerant developed seed, 88 to 100 percent of those developed seed grew. As before, none of the similarly treated vinca developed seed grew, thereby illustrating that non-seminal root growing developed seeds cannot be dried to the same extent as can developed seed from plants that exhibit seminal root growth.

U.S. Pat. Nos. 5,522,907 and No. 5,585,536, whose disclosures are incorporated by reference herein, teach several methods of preparing desiccation-tolerant seed from pregerminated seed. Those methods can also be used to convert desiccation-intolerant developed seed prepared as discussed herein into desiccation-tolerant developed seed.

The developed seed of the present invention can be coated in order to improve its sowability and performance. Many seeds, particularly vegetable seeds, are not uniformly round, which hinders precision planting for optimum crop yields. In other cases, seeds are so small and light that their accurate placement in or on the soil is uncertain. To facilitate the free flow of these seeds in plants, many seed companies provide seeds with coatings of materials that change the shape and size of the seed so that it becomes heavier and rounder. A coated seed, which is frequently referred to as a “pelleted” seed, is characterized by its ability to totally obscure the shape of the encased seed.

Suitable coating materials for use with the developed seed of the present invention include clays such as sub-bentonite and bentonite, vermiculite along with additives such as perlite, pumice, metal stearates, polyethylene, polystyrene, polyurethane, talcum powder, polypropylene, polyvinyl chloride, starches, loams, sugars, arabic gums, organic polymers, celluloses, flours such as wood flours, quartz powders and the like. Additionally, various components can be added to the coating material such as, but not limited to, growth regulators, fungicides, insecticides, safeners and micronutrients. These materials may be added to the developed seeds of the present invention using conventional layering or pelleting procedures that are well known in the seed technology arts. The seed coating described herein can be applied to the developed seed once it is removed from the germination environment.

Other chemicals and methods can be used to interrupt and alter the root development pattern of the seed or somatic embryo in order to obtain the developed seed of the present invention. For example, herbicides, increased levels of ethylene, temperature extremes, pH value extremes, heavy metals, the use of ammonium ions at a high pH values and organic solutes can be used in the germination environment to create the developed seed of the present invention.

The germination environment of the present invention can be used to convert commercially-unusable seed lots into commercially-usable seed lots. More specifically, when commercially-unusable seed is placed into the germination environment of the present invention, commercially-usable, developed seed is obtained. This developed seed can be used in the manner hereinbefore described.

By way of example, and not of limitation, examples of the present invention shall now be given.

### EXAMPLE 1

### Demonstration of Developed Seed

Form in a Wide Variety of Plants

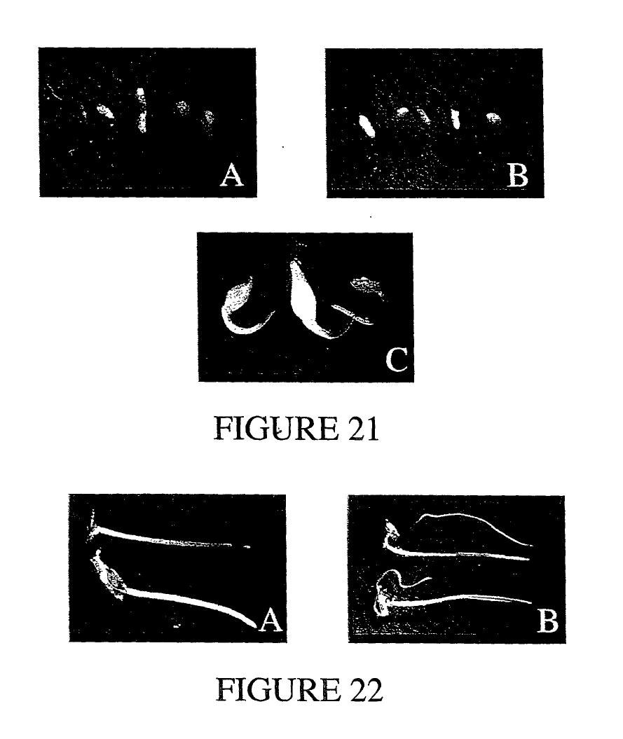

To demonstrate that the precotyledon and cotyledon forms of developed seed could be induced in a wide range of agricultural, horticultural and floricultural crops, seeds from the crops listed below were obtained and germinated in the developed seed solution (and water to provide a visual comparison). The visibly-identifiable features of the precotyledon and cotyledon forms of developed seed (e.g., truncated roots, radial swelling at or near the base of the hypocotyl, shortened hypocotyl, exposed cotyledonary leaves) can be observed in FIGS. 1-24. In one instance, cyclamen somatic embryos treated in the developed seed solution (See FIG. 21) also displayed truncated roots, indicating that even root formation and elongation could be manipulated in in vitro-maintained embryogenic tissue.

Results

### Example 1(a)

### Begonia

Approximately 100 seeds of begonia (variety Cocktail Gin commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/0.5 mM citric acid/0.1 ppm NAA/7 ppm IBA/0.25 mM DTT at 25° C. in a lighted growth room on an orbital shaker. After 7 days, precotyledons and water-treated seedlings were harvested and photographed. After 11 days, cotyledons and water-treated seedlings were harvested and photographed (see FIG. 1).

### Example 1(b)

### Impatiens

Approximately 1,000 seeds of impatiens (variety Dazzler® Cranberry commercially available from Ball Horticultural Company, 622 Town Road, West Chicago, Ill. 60185) were germinated in an aerated column containing either 1 L water or a 1 L solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room. After 6 days, precotyledons and water-treated seedlings were harvested and photographed. After 8 days, cotyledons and water-treated seedlings were harvested and photographed (see FIG. 2).

### Example 1(c)

### Lisianthus

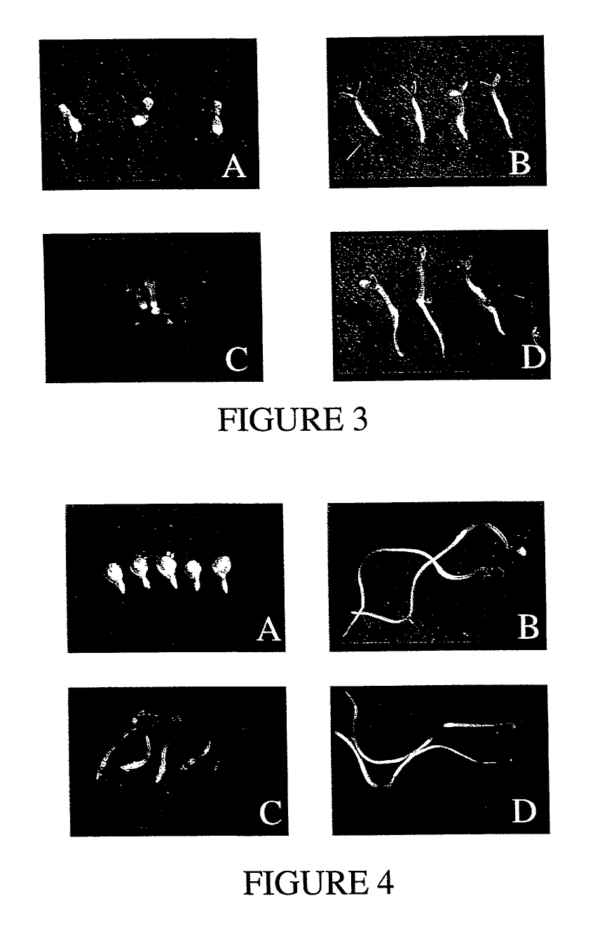

Approximately 100 seeds of lisianthus (variety Lisa Blue commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm AGRONOMIX®/100 ppm KNO₃/0.5 mM citric acid/0.1 ppm NAA/7 ppm IBA/0.25 mM DTT at 25° C. in a lighted growth room on an orbital shaker. After 13 days, precotyledons and water-treated seedlings were harvested and photographed. After 14 days, cotyledons and water-treated seedlings were harvested and photographed (see FIG. 3).

### Example 1(d)

### Petunia

Approximately 100 seeds of petunia (variety Dreams Pink commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/0.5 mM citric acid/8 ppm IBA at 25° C. in a lighted growth room on an orbital shaker. After 5 days, precotyledons and water-treated seedlings were harvested and photographed. After 7 days, cotyledons and water-treated seedlings were harvested and photographed (see FIG. 6).

### Example 1(e)

### Salvia

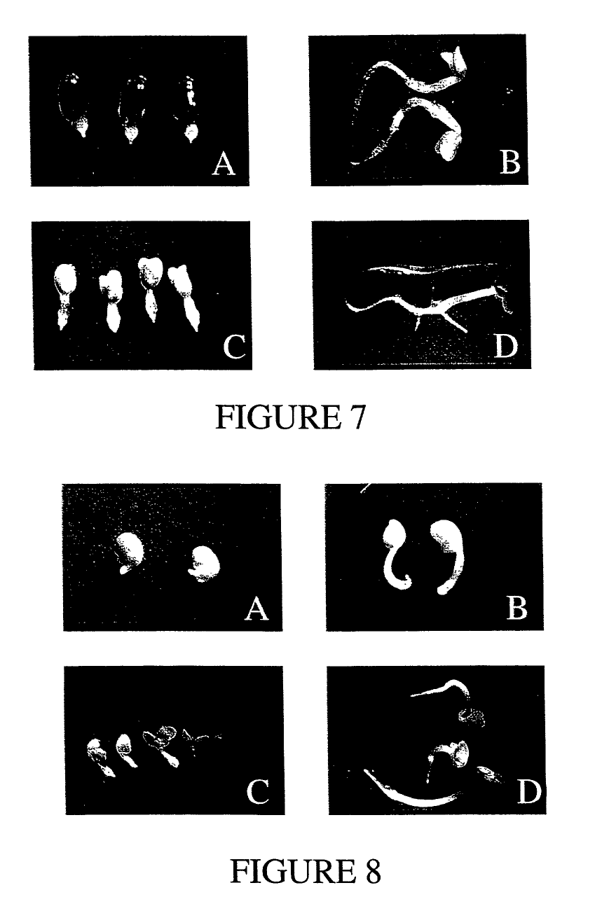

Approximately 100 seeds of salvia (variety Burgundy Vista commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 5 days, precotyledons and water-treated seedlings were harvested and photographed. After 7 days, cotyledons and water-treated seedlings were harvested and photographed (see FIG. 7).

### Example 1(f)

### Stock

Approximately 100 seeds of stock (variety unknown) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 2 days, precotyledons and water-treated seedlings were harvested and photographed. After 4 days, cotyledons and water-treated seedlings were harvested and photographed (see FIG. 8).

### Example 1(g)

### Verbena

Approximately 100 seeds of verbena (variety Quartz Burgundy commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/0.5 mM citric acid/8 ppm IBA at 25° C. in a lighted growth room on an orbital shaker. After 6 days, precotyledons and water-treated seedlings were harvested and photographed (see FIG. 9).

### Example 1(h)

### Vinca

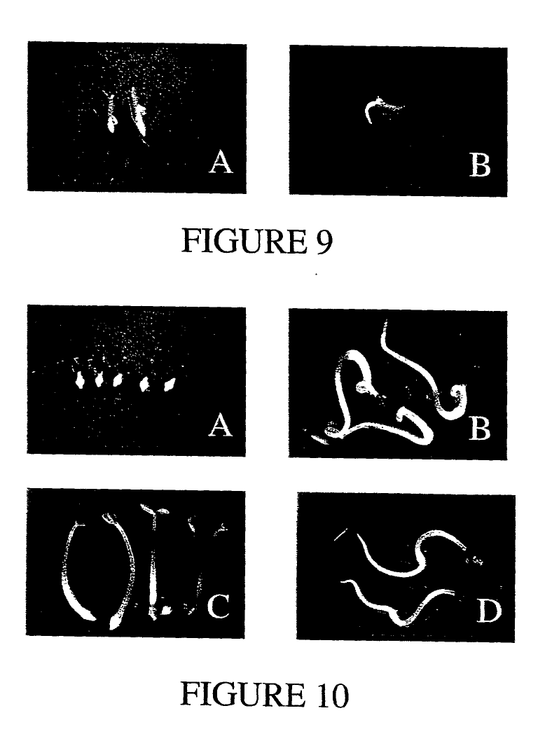

Approximately 100 seeds of vinca (variety Coconut Cooler commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/0.5 mM citric acid/8 ppm IBA at 25° C. in a lighted growth room on an orbital shaker. After 6 days, precotyledons and water-treated seedlings were harvested and photographed. After 11 days, cotyledons and water-treated seedlings were harvested and photographed (see FIG. 10).

### Example 1(i)

### Broccoli

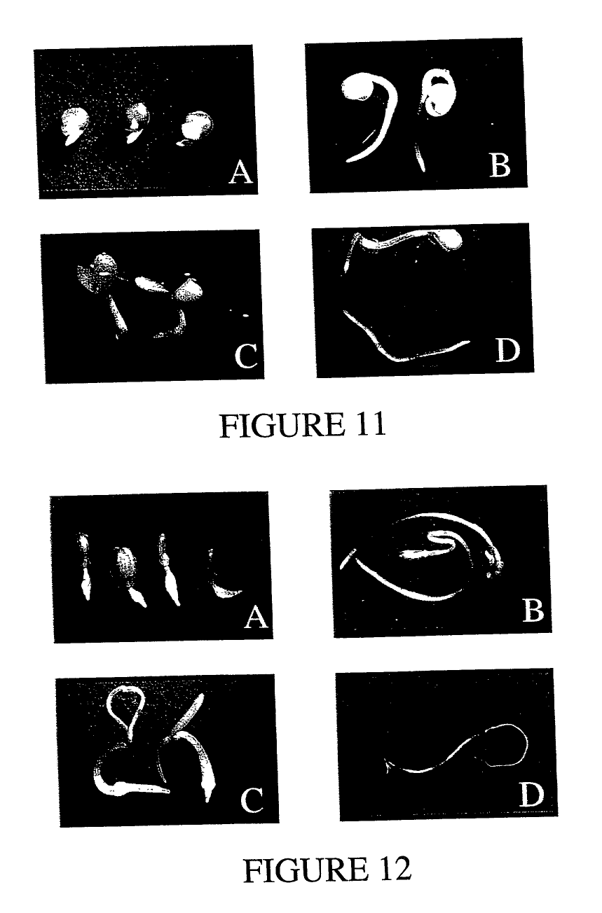

Approximately 100 seeds of broccoli (variety Packman commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 2 days, precotyledons and water-treated seedlings were harvested and photographed. After 3 days, cotyledons and water-treated seedlings were harvested and photographed (see FIG. 11).

### Example 1(j)

### Carrot

Approximately 100 seeds of carrot (variety Chantenay Red Cored commercially available from NK Lawn & Garden Company, P.O. Box 24028, Chattanooga, Tenn. 37422) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 5 days, precotyledons and water-treated seedlings were harvested and photographed. After 10 days, cotyledons and water-treated seedlings were harvested and photographed (see FIG. 12).

### Example 1(k)

### Cauliflower

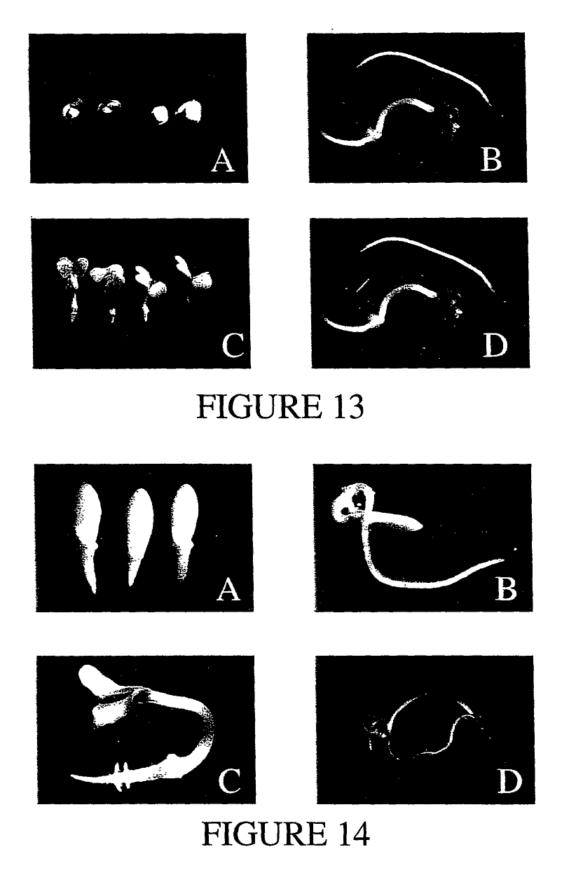

Approximately 100 seeds of cauliflower (variety Snowball Y Improved commercially available from NK Lawn & Garden Company, P.O. Box 24028, Chattanooga, Tenn. 37422) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 4 days, precotyledons and water-treated seedlings were harvested and photographed. After 4 days, cotyledons and water-treated seedlings were harvested and photographed (see FIG. 13).

### Example 1(l)

### Cucumber

Approximately 50 seeds of cucumber (variety Straight Eight commercially available from NK Lawn & Garden Company, P.O. Box 24028, Chattanooga, Tenn. 37422) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 4 days, precotyledons and water-treated seedlings were harvested and photographed. After 10 days, cotyledons and water-treated seedlings were harvested and photographed (see FIG. 14).

### Example 1(m)

### Lettuce

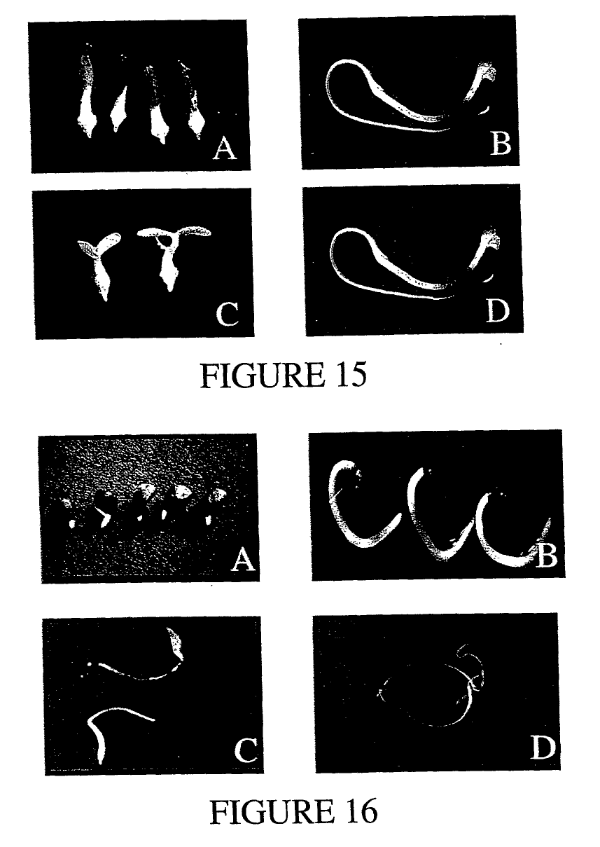

Approximately 100 seeds of lettuce (variety Grand Rapids commercially available from NK Lawn & Garden Company, P.O. Box 24028, Chattanooga, Tenn. 37422) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 4 days, precotyledons and water-treated seedlings were harvested and photographed. After 4 days, cotyledons and water-treated seedlings were harvested and photographed (see FIG. 15).

### Example 1(n)

### Onion

Approximately 100 seeds of onion (variety Redman commercially available from W. Atlee Burpee & Company, 300 Park Avenue, Warminster, Pa. 18974) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 4 days, precotyledons and water-treated seedlings were harvested and photographed. After 11 days, cotyledons and water-treated seedlings were harvested and photographed (see FIG. 16).

### Example 1(o)

### Pepper

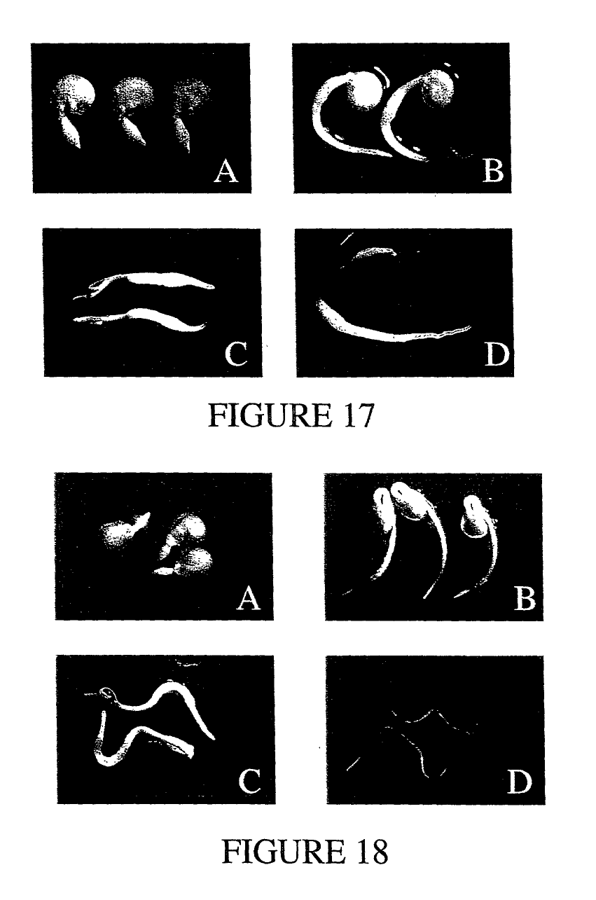

Approximately 100 seeds of pepper (variety Better Belle commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.25 ppm NAA/7 ppm IBA/0.25 mM DTT at 25° C. in a lighted growth room on an orbital shaker. After 6 days, precotyledons and water-treated seedlings were harvested and photographed. After 12 days, cotyledons and water-treated seedlings were harvested and photographed (see FIG. 17).

### Example 1(p)

### Tomato

Approximately 100 seeds of a tomato variety called “Tumbler” (commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 3 days, precotyledons and water-treated seedlings were harvested and photographed (FIG. 18). After 6 days, cotyledons and water-treated seedlings were harvested and photographed (FIG. 18).

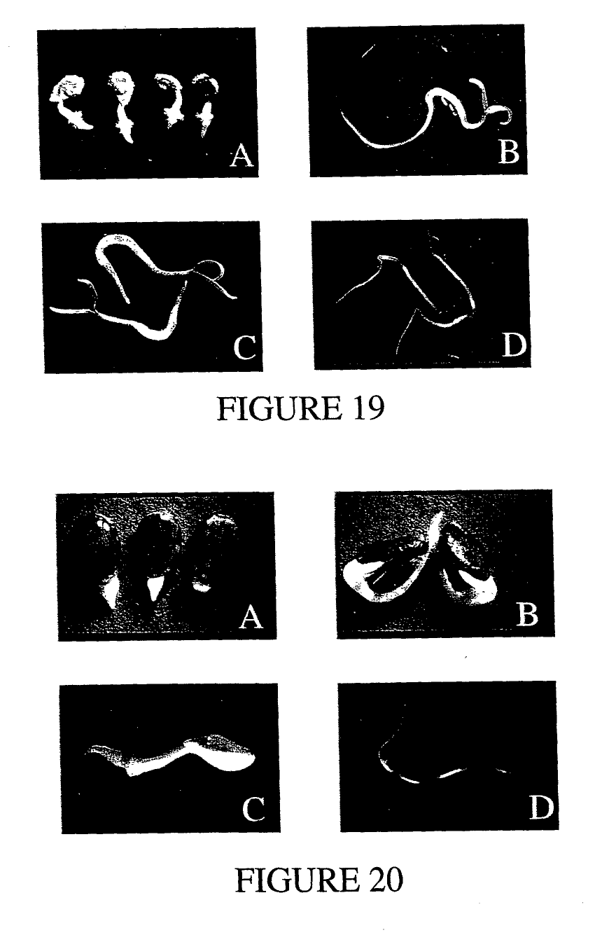

Approximately 100 seeds of a tomato variety called “Beefmaster” (commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.25 ppm NAA/7 ppm IBA/0.25 mM DTT at 25° C. in a lighted growth room on an orbital shaker. After 6 days, precotyledons and water-treated seedlings were harvested and photographed (FIG. 19). After 7 days, cotyledons and water-treated seedlings were harvested and photographed (FIG. 19).

### Example 1(q)

### Watermelon

Approximately 25 seeds of watermelon (variety Crimson Sweet commercially available from NK Lawn & Garden Company, P.O. Box 24028, Chattanooga, Tenn. 27422) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room on an orbital shaker. After 5 days, precotyledons and water-treated seedlings were harvested and photographed. After 12 days, cotyledons and water-treated seedlings were harvested and photographed (see FIG. 20).

### Example 1(r)

### Cyclamen Somatic Embryos

Cyclamen somatic embryos of line #003 (an experimental variety of Ball Horticultural Company, 622 Town Road, West Chicago, Ill. 60185), ranging in size from 0.5-1 mm in diameter were produced using standard procedures. These 3-week old embryos were germinated in 125 mL Erlenmeyer flasks containing either 50 mL B₅ S₂₀ germination medium (with 2% sucrose) or a 50 mL solution of 100 ppm Peters Fertilizer/100 ppm KNO₃/100 ppm AGRONOMIX®/0.5 mM citric acid/0.25 ppm NAA/7 ppm IBA/2% sucrose at 25° C. in a lighted growth room on an orbital shaker. After 11 days, the somatic embryos were harvested and photographed (see FIG. 21).

### Example 1(s)

### Turfgrass

Approximately 100 seeds of Kentucky Bluegrass (commercially available from Franks Nursery & Crafts, 1175 W. Long Lake, Troy, Mich. 48098) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.25 ppm NAA/7 ppm IBA/0.25 mM DTT at 25° C. in a lighted growth room on an orbital shaker. After 7 days, cotyledons and water-treated seedlings were harvested and photographed (see FIG. 22).

### Example 1(t)

### Rice

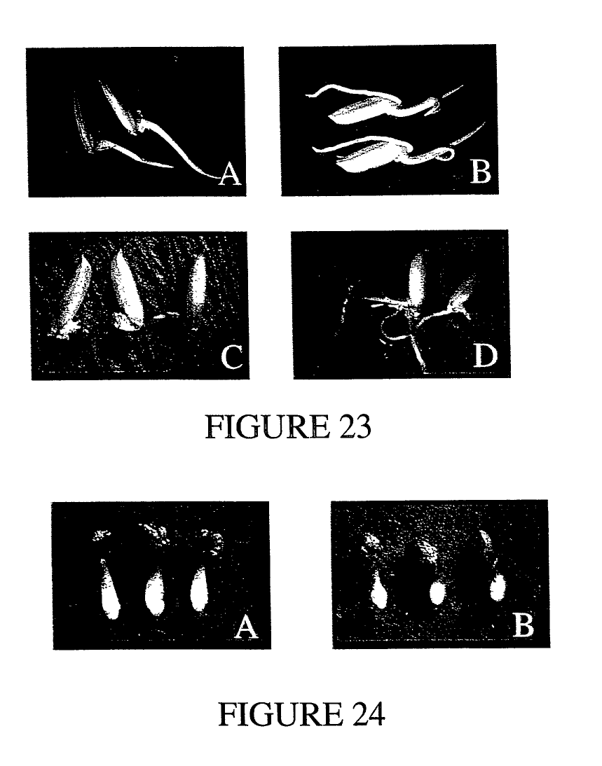

Approximately 100 seeds of rice (variety Cypress obtained from Louisiana State University (hereinafter ALSU@) Rice Research Station) were germinated in 125 mL Erlenmeyer flasks containing either 40 mL water or a 40 mL solution of 100 ppm Peters Fertilizer/100 ppm KNO₃/0.5 mM citric acid/8 ppm IBA at 25° C. in a lighted growth room on an orbital shaker. After 4 days, cotyledons and water-treated seedlings were harvested and photographed (see FIG. 23).

### Example 1(u)

### Cyclamen

Approximately 1,000 seeds of cyclamen (variety Royal Scarlet commercially available from Ball Horticultural Company, 622 Town Road, West Chicago, Ill. 60185) were germinated in an aerated column containing either 3 L water or a 3 L solution of 100 ppm Peters Fertilizer/100 ppm KNO₃/100 ppm AGRONOMIX®/0.5 mM citric acid/0.1 ppm NAA/7 ppm IBA at 15° C. in a lighted growth room. After 35 days, precotyledon seedlings were harvested and photographed (see FIG. 24).

### EXAMPLE 2

### Conversion of Pregerminated Seeds into Developed Seeds of the Present Invention

U.S. Pat. Nos. 4,905,411, No. 5,522,907 and No. 5,585,536 disclose pregerminated seeds having emerged radicles and a moisture content at which radicle development is suspended without a loss of seed viability.

To demonstrate that the final developmental stage of seed germination achieved in the pregerminated seeds described in U.S. Pat. Nos. 4,905,411, 5,522,907 and 5,585,536 occurs much earlier than the developed seeds of the present invention, and can be developmental precursors to the developed seeds described herein, the following studies were performed with primed and pregerminated pansy seeds. It was reasoned that if the primed and pregerminated pansy seeds were developmentally more advanced than the developed seeds of the present invention, then the precotyledon and cotyledon forms would not be recovered. If, however, the primed and pregerminated pansy seeds were developmental precursors to the developed seed forms of the present invention, then both precotyledon and cotyledon forms would be recovered.

Primed Pansy Seeds

Approximately 100 primed seeds of pansy (variety Baby Bingo Sky Blue commercially available from Ball Horticultural Company, 622 Town Road, West Chicago, Ill. 60185) were germinated in 125 mL Erlenmeyer flasks containing either 40 mL water or a 40 mL solution of 100 ppm Peters Fertilizer/100 ppm KNO₃/0.5 mM citric acid/8 ppm IBA at 25° C. in a lighted growth room on an orbital shaker. After 6 days, precotyledons and water-treated seedlings were harvested and photographed. After 8 days, cotyledons and water-treated seedlings were harvested and photographed (shown in FIG. 4).

Pregerminated Pansy Seeds

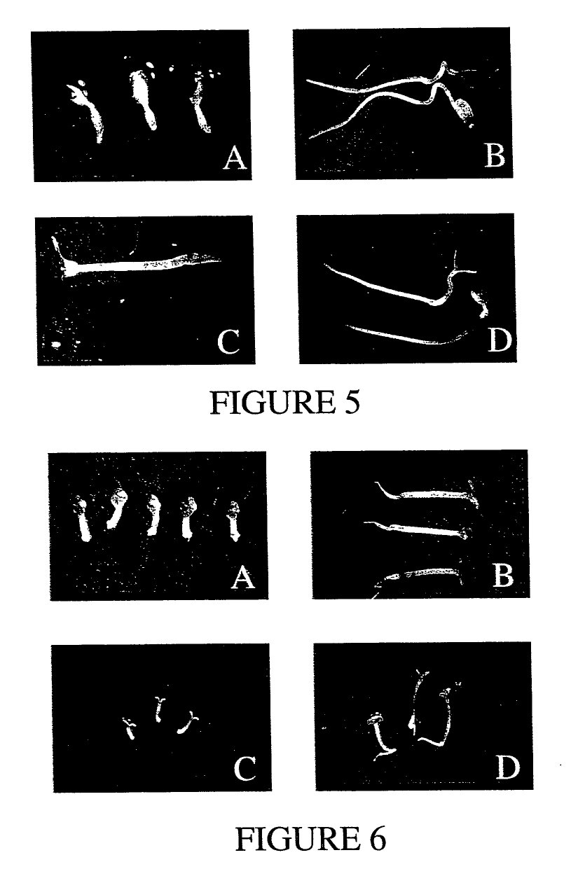

Approximately 100 pregerminated seeds of pansy (variety Delta Pure commercially available from Novartis Seed, Inc. Flowers, 5300 Katrine Avenue, Downers Grove, Ill. 60515) were germinated in 125 mL Erlenmeyer flasks containing either 40 mL water or a 40 mL solution of 100 ppm Peters Fertilizer/100 ppm KNO₃/100 ppm AGRONOMIX®/0.5 mM citric acid/0.25 ppm NAA/7 ppm IBA/0.25 mM DTT at 25° C. in a lighted growth room on an orbital shaker. After 5 days, precotyledons and water-treated seedlings were harvested and photographed. After 11 days, cotyledons and water-treated seedlings were harvested and photographed (shown in FIG. 5).

Results

FIG. 4 shows the precotyledon and cotyledon forms of pansy developed seed of the present invention that were derived from primed pansy seed. The pansy precotyledon displays a dramatically truncated root and basal radial swelling, features that are totally lacking in the water-treated seeds. The truncated root of the cotyledon form is clearly visible. These results demonstrate that primed pansy seeds can be induced to develop into precotyledon and cotyledon forms, and thus represent developmental precursors to the developed seed of the present invention.

FIG. 5 shows the precotyledon and cotyledon forms of pansy developed seed that were derived from pregerminated pansy seed. As can be clearly observed, the pregerminated pansy seeds can be induced to yield the same precotyledon and cotyledon forms as were produced with the primed pansy seeds in FIG. 4. These results clearly demonstrate that the pregerminated seeds can be induced to develop into precotyledons and cotyledons, and thus are developmentally less advanced than either the precotyledon or cotyledon form of developed seed form described in the present invention.

Taken together, the results demonstrate the successful utilization of pregerminated pansy seed (the finalized seed form described in U.S. Pat. Nos. 4,905,411, No. 5,522,907 and No. 5,585,536) as the starting seed material to obtain the pansy precotyledon and cotyledon forms using the methods described herein and provide strong evidence for the advanced developmental stages achieved in pansy developed seed.

### EXAMPLE 3

### The Impact of Calcium Utilization on Root Elongation in Germinating Seeds

To investigate other mechanisms that might disrupt calcium utilization by germinating seeds leading to adverse effects on root formation and elongation, a number of known calcium effectors were analyzed. EGTA is a well-known calcium-chelating compound which binds free calcium ions. The element, manganese, has been characterized to act as a calcium channel blocker in the cellular environment of the plasma membrane, thereby inhibiting uptake and utilization of calcium by the cell. Finally, auxins, like IBA, can disrupt calcium signaling pathways in the cell, thereby disrupting cellular developmental processes like rooting. For these experiments, seeds were germinated in the presence of these compounds, and the lengths of the roots (in mm) from 20 randomly-selected seeds (per treatment) were measured. These values were then compared to the lengths of the roots from 20 randomly-selected seeds germinated in water during the same time period and under the same environmental conditions.

### Example 3(a)

### Impatiens

Approximately 100 seeds of impatiens (variety Dazzler® Pink commercially available from Ball Horticultural Company, 622 Town Road, West Chicago, Ill. 60185) were germinated in 125 mL Erlenmeyer flasks containing either 40 mL water, 6 mM EGTA, 5 mM manganese chloride or 8 ppm IBA at 25° C. in a lighted growth room on an orbital shaker. After 5 days, root lengths were measured and recorded (see Table 1 below). As can be observed, the water-treated seeds developed roots averaging 6.9 mm in length. In contrast, root growth was totally inhibited by exposure to EGTA and IBA. Some minor root growth was evidenced on the manganese chloride-treated seeds (see Table 1 below).

### Example 3(b)

### Pansy

Approximately 100 primed seeds of pansy (variety Baby Bingo Sky Blue commercially available from Ball Horticultural Company, 622 Town Road, West Chicago, Ill. 60185) were germinated in 125 mL Erlenmeyer flasks containing either 40 mL water, 6 mM EGTA or 5 mM manganese chloride at 25° C. in a lighted growth room on an orbital shaker. After 5 days, root lengths were measured and recorded (see Table 1 below). Although the majority of the water-treated seeds displayed roots ranging in length from 7-15 mm (an average of 10.3 mm), the manganese chloride-treated seeds were inhibited by 70-90%, and the EGTA-treated seeds by an even greater extent (see Table 1 below).

### Example 3(c)

### Vinca

Approximately 100 seeds of vinca (variety Coconut Cooler commercially available from Ball Horticultural Company, 622 Town Road, West Chicago, Ill. 60185) were germinated in 125 mL Erlenmeyer flasks containing either 40 mL water, 6 mM EGTA or 5 mM manganese chloride at 25° C. in a lighted growth room on an orbital shaker. After 5 days, root lengths were measured and recorded (see Table 1 below). For water-treated seeds, root lengths averaged approximately 12 mm in length. However, the manganese-treated seeds averaged only 3 mm while the EGTA-treated seeds exhibited roots of less than 1 mm (see Table 1 below).

### Example 3(d)

### Pepper

Approximately 100 seeds of pepper (variety Holiday Flame commercially available from Ball Horticultural Company, 622 Town Road, West Chicago, Ill. 60185) were germinated in 125 mL Erlenmeyer flasks containing either 40 mL water, 6 mM EGTA or 5 mM manganese chloride at 25° C. in a lighted growth room on an orbital shaker. After 5 days, root lengths were measured and recorded (see Table 1 below). As can be observed, root lengths averaging 6.5 mm were typical of water-treated seeds. However, both EGTA and manganese inhibited root elongation, with EGTA being more efficacious than manganese (see Table 1 below).

### Example 3(e)

### Tomato

Approximately 100 seeds of tomato (variety Tumbler commercially available from Ball Horticultural Company, 622 Town Road, West Chicago, Ill. 60185) were germinated in 125 mL Erlenmeyer flasks containing either 40 mL water, 6 mM EGTA or 5 mM manganese chloride at 25° C. in a lighted growth room on an orbital shaker. After 5 days, root lengths were measured and recorded (see Table 1 below). Like pepper, the water-treated seeds displayed roots averaging 6.5 mm in length. Modest inhibition of root elongation was observed with manganese treatment. However, root elongation was significantly inhibited by exposure to EGTA (see Table 1 below).

### Example 3(f)

### Rice

Approximately 100 seeds of rice (variety Cypress obtained from LSU Rice Research Station) were germinated in 125 mL Erlenmeyer flasks containing either 40 mL water, 6 mM EGTA or 5 mM manganese chloride at 25° C. in a lighted growth room on an orbital shaker. After 5 days, root lengths were measured and recorded (see Table 1 below). The water-treated seeds developed exceptionally long roots averaging 15.6 mm in length. Interestingly, manganese had no effect on root elongation while EGTA inhibited root elongation by up to nearly 80% (see Table 1 below).

TABLE 1

| | Treatment | | | |
|---|---|---|---|---|
| Crop and Variety | Water | EGTA | Manganese | IBA |
| Impatiens Dazzler ® Pink | 6.9¹ | 0.0 | 0.2 | 0.0 |
| Pansy Baby Bingo Sky Blue | 10.3 | 0.4 | 1.9 | ND² |
| Vinca Coconut Cooler | 12.3 | 0.8 | 3.0 | ND |
| Pepper Holiday Flame | 6.5 | 1.8 | 4.2 | ND |
| Tomato Tumbler | 6.5 | 1.8 | 4.4 | ND |
| Rice Cypress | 15.6 | 3.2 | 15.6 | ND |

¹Root length (in mm).
²Not Determined

### EXAMPLE 4

### Conversion of Commercially-Unusable Seed Lots into Commercially-Usable Seed Lots

In floricultural, horticultural and agricultural growing practices today, a high priority is placed upon the commercial availability of high-quality seed lots. The goal for the growers of many floricultural and horticultural crops is 100% usable plants from a particular seed lot. In most cases, this lofty goal is not attained, and growers must be satisfied with only 70-90% usable plants. In some instances, even yields in that range cannot be achieved, and those seed lots are labeled as commercially unfit for sale to growers. Hence, the breeder/producer entity or producer entity must absorb this financial loss. If these seed lots could be converted from an unusable seed lot into a usable one, an economic benefit would be realized. This Example demonstrates that poor quality seed lots of impatiens and vinca (i.e., low germination rates) can be readily converted into high-quality seed lots. Impatiens and vinca precotyledon forms are produced, separated from ungerminated seeds by physical methods known in the art, and then sown. These precotyledon forms of developed seed now yield 90-100% usable plants, or nearly twice the percentage observed for sown raw seed.

### Example 4(a)

### Vinca Lot Recovery

Approximately 1,000 seeds of vinca (see varieties below, which are all commercially available from Ball Horticultural Company, 622 Town Road, West Chicago, Ill. 60185) were germinated for 4 days in aerated columns in 1 L 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted environmental room. Precotyledon forms were harvested and were enriched for on the basis of their buoyant density by methods known in the art and then sown in germination boxes (20 per box). Raw seed of each of the vinca varieties was also sown (20 per box) on the same day as the precotyledon forms. All boxes were then maintained at 25° C. in a lighted environmental chamber. Percent yield indicates the percentage (where 100%=20) of seeds which continued normal seedling growth and development after sowing. Data was collected 11 days after sowing and the results are shown below in Table 2.

TABLE 2

Percent Yield

| Vinca Variety | | Raw Seed | Developed Seed |
|---|---|---|---|
| 1) | Peppermint Improved | 45% | 95% |
| | Cooler | | |
| 2) | Strawberry | 45% | 100% |
| | Cooler | | |
| 3) | Blush | 60% | 100% |
| | Cooler | | |
| 4) | Coconut | 60% | 100% |
| | Cooler | | |
| 5) | Icy Pink | 70% | 100% |
| | Cooler | | |
| 6) | Pink | NS¹ | 100% |
| | Cooler | | |
| 7) | Grape | 40% | 100% |
| | Cooler | | |

¹Not sown

### Example 4(b)

### Impatiens Lot Recovery

Approximately 2,000 seeds of impatiens (variety Swirl Cherry commercially available from Ball Horticultural Company, 622 Town Road, West Chicago, Ill. 60185) were germinated for 7 days in an aerated column in 1 L 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted environmental room. Precotyledon forms were harvested and enriched for on the basis of their buoyant density by methods known in the art and then sown in a germination box (20 per box) or a plug tray (56 each). Raw seed of Swirl Cherry (identical numbers as the precotyledon forms) were also sown on the same day as the developed seed. Boxes were then maintained at 25° C. in a lighted environmental chamber while plug trays were maintained under Stage 2 conditions. Percent yield indicates the percentage of seeds sown that continued normal seedling growth and development after sowing. Data was collected 14 days after sowing and the results shown below in Table 3.

TABLE 3

Percent Yield

| | | |
|---|---|---|
| Variety | Raw Seed | Developed Seed |

GERMINATION BOXES

| | | |
|---|---|---|
| Swirl Cherry | 51% | 93% |

PLUG TRAYS

| | | |
|---|---|---|
| Swirl Cherry | 51% | 90% |

### EXAMPLE 5

### Compact Phenotype of Plants Grown from Developed Seed

To demonstrate that impatiens seedlings derived from the precotyledon form of developed seed have a more compact phenotype due to shorter internodes (as compared to seedlings started with raw seed), the following experiment was performed.

Approximately 1,000 seeds of impatiens (varieties Dazzler® Cranberry and Super Elfin® White, both commercially available from Ball Horticultural Company, 622 Town Road, West Chicago, Ill. 60185) were germinated in an aerated column containing a 1 L solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room. After 5-6 days treatment, precotyledon forms were harvested and sown on soilless plug-growing media (2 trays per variety). On the same day that the precotyledon forms were sown, raw seed of these two varieties was also sown (2 trays per variety). All trays were maintained in the greenhouse and fertilized on a weekly basis with 75 ppm 20-10-20 liquid fertilizer. After approximately two weeks in the greenhouse, 25 representative seedlings were selected from each tray and the plant height was measured (Day 0). On the same day that the height measurements were recorded, one tray of young plants sown with raw seed was sprayed with an application of the growth regulator, B-Nine® (2500 ppm) (Uniroyal Chemical Company (Akron, Ohio)). After an additional 6 days, 25 representative seedlings from each tray were selected and the plant height measured again. These measurements are shown below in Table 4.

On the day of growth regulator application (after two weeks in the greenhouse), the Dazzler® Cranberry young plants derived from the raw seed were slightly taller than the precotyledon-derived seedlings (3.2 and 3.7 cm versus 2.9 and 2.9 cm). These differences were less obvious in the Super Elfin® white plants (2.4 and 2.5 cm versus 2.0 and 2.4 cm), in large part because this variety is inherently more compact than the Dazzler® Cranberry variety. However, in spite of these apparent minor differences, the observation that the raw seed-derived Dazzler® Cranberry and Super Elfin® White young plants were as tall or even taller than the precotyledon-derived plants is very significant, considering that the precotyledon forms (and derived plants) were far more developmentally advanced than the raw seed (and derived plants) at the time that they were sown and moved to the greenhouse. Therefore, it could be reasoned that the growth rate of the precotyledon-derived seedlings must be significantly more controlled compared to the raw seed-derived plants to have caused this outcome. This observation is supported by the results of the following study.

Six days after application of the growth regulator, the untreated seedlings derived from raw seed of both varieties clearly were the tallest young plants. The Dazzler® Cranberry and Super Elfin® White untreated seedlings (raw, untreated) averaged 6.7 and 4.4 cm in height, respectively. In contrast, the seedlings grown from raw seed that had been treated with growth regulator (raw, treated) averaged 5.4 and 3.2 cm for Dazzler® Cranberry and Super Elfin® White, respectively, over a 1 cm reduction in height for both varieties. This 20% and 27% reduction in height for Dazzler® Cranberry and Super Elfin® White, respectively, indicated that these varieties both responded similarly to the application of growth regulator. By comparison, the young plants of both varieties grown from precotyledon forms were shorter than the raw seed-derived, untreated plants. The Dazzler® Cranberry precotyledon-derived plants averaged 4.8 and 5.4 cm for each tray, both shorter than the raw seed-derived untreated plants (6.7 cm), and very similar to the raw seed-derived plants treated with growth regulator (5.4 cm). For Super Elfin® White, the precotyledon-derived plants averaged 3.0 and 3.7 cm in height for each tray. These values are less than the raw seed-derived, untreated young plants (4.4 cm), and very similar to the 3.2 cm average height observed for the raw seed-derived plants treated with growth regulator.

If the growth that occurred among the various trays of young plants during the 6-day period immediately following the application of growth regulator to one tray of raw seed-derived plants is compared, the height control exhibited by the precotyledon-derived plants becomes even more obvious. The Dazzler® Cranberry raw seed-derived plants (untreated) grew an average of 3.5 cm during that period. In contrast, the growth regulator-treated (raw seed-derived) seedlings only grew an average of 1.7 cm (49% of the untreated). It can be observed that the two trays of precotyledon-derived seedlings grew an average of 1.9 cm (54% of untreated) and 2.5 cm (71% of untreated), amounts well below the value for the untreated seedlings.

Likewise, the Super Elfin® White raw seed-derived plants grew an average of 2.0 cm during that period. In contrast, the growth regulator-treated seedlings only grew an average of 0.8 cm (40% of untreated). By comparison, it can be observed that the precotyledon-derived seedlings grew 1.1 cm (55% of untreated) and 1.2 cm (60% of untreated), amounts well below that observed for the untreated seedlings, and similar to that observed for the growth regulator-treated seedlings.

These results demonstrate that the precotyledon-derived plants have a modified growth pattern and habit compared to the raw seed-derived plants. More specifically, the developed seed-derived plants display a reduced internode length. Early in the experiment, the precotyledon-derived seedlings were considerably more developmentally advanced than the raw seed-derived plants. This was easily monitored by comparing the number and size of true leaves on the individual seedlings. However, during the first several weeks in the greenhouse, the height of the raw seed-derived plants eventually equals or surpasses the height of the precotyledon-derived plants while being no more developmentally advanced (based upon the number and sizes of true leaves). The rate of height increase in the precotyledon-derived plants most closely matches that of raw seed-derived plants that have been treated with an application of growth regulator like B-Nine® (Uniroyal Chemical Company (Akron, Ohio)). Taken together, these results demonstrate that precotyledon-derived young plants display a modified growth rate and pattern which yields young plants which are more compact, are “toned” and display the highly desirable phenotype of “hardened” plugs. Most importantly, these results further demonstrate that the method used to create developed seed has a significant impact on the development of the seedlings that are derived from these developed seed.

TABLE 4

| | DAY 0 | DAY 6 | DAY 0-6 |
|---|---|---|---|
| Impatiens Variety | Growth | Growth | Growth |
| Dazzler ® Cranberry-Raw, | 3.2¹ | 6.7¹ | 3.5² |
| Untreated | | | |
| Dazzler ® Cranberry-Raw, | 3.7 | 5.4 | 1.7 |
| Treated | | | |
| Dazzler ® Cranberry- | 2.9, | 4.8, | 1.9, |
| Developed Seed | 2.9 | 5.4 | 2.5 |
| Super Elfin ® White-Raw, | 2.4 | 4.4 | 2.0 |
| Untreated | | | |
| Super Elfin ® White-Raw, | 2.5 | 3.2 | 0.7 |
| Treated | | | |
| Super Elfin ® White- | 2.0, | 3.0, | 1.0, |
| Developed Seed | 2.4 | 3.7 | 1.3 |

¹Plant height (in cm).
²Difference in growth between Day 0 and Day 6.

### EXAMPLE 6

### Reduction of Hypocotyl Length in Developed Seed from Vinca Precotyledons

To demonstrate that vinca seedlings derived from the precotyledon form of developed seed have a more compact phenotype due to reduced hypocotyl length (as compared to seedlings started with raw seed), the following study was performed.

Approximately 1,000 seeds of vinca (varieties Blush Cooler, Grape Cooler, Icy Pink Cooler, Peppermint Improved Cooler and Strawberry Cooler, all commercially available from Ball Horticultural Company, 622 Town Road, West Chicago, Ill. 60185) were germinated in an aerated column containing a 1 L solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room. After 4 days treatment, the precotyledon forms were harvested and sown on moist blotter paper in germination boxes for incubation at 25° C. in a lighted growth chamber. On the same day that the precotyledon forms were sown, raw seeds of these same five varieties were similarly sown and maintained under identical conditions. After approximately one month, the hypocotyl lengths of the precotyledon-form and raw seed-derived seedlings were measured.

As can be observed below in Table 5, the hypocotyls were shorter in length for all seedlings derived from the precotyledon forms compared to those from raw seed. For Icy Pink Cooler, the average hypocotyl lengths for the raw seed and developed seed were 2.3 and 1.3 cm, respectively, a reduction in length of 43%. The results for Strawberry Cooler were almost identical (raw and developed seed were 2.4 and 1.3 cm, respectively, a reduction of 46%). The results for Blush Cooler and Peppermint Improved Cooler were virtually identical. The raw seed-derived seedlings from both varieties were 1.9 cm in height while the developed seed-derived seedlings were 1.0 and 0.8 cm, for Blusher Cooler and Peppermint Improved Cooler, respectively. Finally, Grape Cooler showed the most dramatic reduction in hypocotyl length as the precotyledon-derived seedlings were reduced in height by 74% (from 2.7 cm to 0.7 cm). Taken together these results demonstrate that developed seed-derived vinca seedlings exhibit reduced hypocotyl stretch compared to raw seed-derived vinca seedlings. These observations support the conclusions reached regarding the compact young plant habit observed in developed seed-derived impatiens plants.

TABLE 5
Seedling Hypocotyl Length (in cm)

| | | Developed | Percent |
|---|---|---|---|
| Vinca Variety | Raw Seed | Seed | Reduction |
| Blush Cooler | 1.9 | 1.0 | 47 |
| Grape Cooler | 2.7 | 0.7 | 74 |
| Icy Pink Cooler | 2.3 | 1.3 | 43 |
| Peppermint | 1.9 | 0.8 | 58 |
| Improved Cooler | | | |
| Strawberry | 2.4 | 1.3 | 46 |
| Cooler | | | |

### EXAMPLE 7

### Enhanced Rooting of Developed Seed

The following studies demonstrate that the precotyledon forms of impatiens developed seed not only proliferate an extensive network of root tissue in an expedient manner when sown in a suitable environment, but that the root architecture has been modified in a way such that secondary roots appear in increased numbers much earlier than is normally found in germinating raw seed.

Approximately 1,000 seeds of impatiens (varieties Dazzler® Cranberry and Super Elfin® Red are commercially available from Ball Horticultural Company, 622 Town Road, West Chicago, Ill. 60185) were germinated in an aerated column containing a 1 L solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room. After 5 days in the germination solution, the precotyledon forms were harvested and 25 of each variety were sown in germination boxes and maintained at 25° C. in a lighted growth chamber. Raw seeds (25 of each variety) of these same two varieties were also similarly sown on the same day and maintained under identical conditions.

Three days after sowing, the impatiens seedlings were photographed with a CCD camera, and the total root area calculated using Quantimet Image Processing Software (hereinafter “QUIPS”) as described in U.S. Pat. Nos. 5,659,623 and No. 5,572,827, herein incorporated by reference. Dazzler® Cranberry precotyledon-derived seedlings produced a total root area of 531.9 mm². By comparison, the Dazzler® Cranberry raw seed-derived seedlings yielded only 77.1 mm² of root tissue, only 14% of that observed for those from developed seeds. Similarly, the Super Elfin® Red seedlings from raw seeds produced only 16.4 mm² of root tissue. In contrast, developed seed-derived seedlings of the same variety generated 293.5 mm² of root tissue, an 18-fold greater area. These results taken together convincingly demonstrate that impatiens developed seed exhibit greatly enhanced root development as compared to the raw impatiens seed three days after sowing in a suitable environment.

Upon closer examination of the root architecture, it can be observed that the majority of the root area in the raw seed is contributed by the primary root and to a much, much lesser extent by the secondary roots. In contrast, the large majority of the root area observed in the developed seed-derived seedlings is contributed by the secondary roots, and not the primary root. In fact, the primary root in some of the impatiens developed seeds does not continue to elongate, and root development occurs primarily among the secondary roots (up to 5-6 per seedling) which originate from the region of the developed seed exhibits which the basal radial swelling.

In an experiment identical to that immediately described above, Dazzler® Cranberry and Super Elfin® Red precotyledon forms of developed seed were sown in germination boxes and the number of secondary roots present determined after three days for each of the 25 seedlings. The Super Elfin® Red and Dazzler® Cranberry precotyledon-derived seedlings displayed an average of 4.2 and 4.6 secondary roots per seedling, respectively. In sharp contrast, germinating raw seeds of either variety generally exhibited only a single (one) primary root at this stage of growth and development. These results taken together help provide a scientific explanation for the enhanced proliferation of root mass which occurs after sowing of impatiens developed seeds.

### EXAMPLE 8

### Post-Harvest Handling of Developed Seed: Optimization of Storage Conditions

Various temperatures and cooling regimes were tested for their ability to inhibit continued seedling development while maintaining viability of the germinated developed seeds. Impatiens Dazzler® Red and Dazzler® White were first primed in an osmotic solution containing PEG-8000 (-8 bar), and then placed into an aerated column containing the developed seed solution at 25° C. in a lighted growth chamber. When the first population of seeds had attained the desired precotyledon form of developed seed, the entire column of seeds was harvested, purified, rinsed, de-watered and placed into plastic, sealable storage vials at the temperatures detailed below. After one (1) week at the indicated storage temperature, a portion of the seeds (three (3) replicates of twenty (20) seeds each) was removed and sown in germination boxes to monitor continued seedling development, as evidenced by resumed root elongation and root hair proliferation within (2) days after sowing.

The various temperature treatments tested were: (i) directly moved to 5° C.; (ii) treated at 35° C. for 2 hours followed by cooling to 5° C. over six (6) hours and maintained at 5° C.; (iii) cooling from 25° C. to 5° C. over eighteen (18) hours and maintained at 5° C.; and (iv) room temperature storage. The results are shown below in Table 6. For Dazzler® Red, the poorest response was observed when the developed seeds were directly placed at 5° C. as only twelve percent (12%) resumed root elongation within forty-eight (48) hours after sowing. Similarly, the six (6) hour cool-down period to 5° C. and storage at 5° C. only slightly improved rates (28%). The best rate was observed when the cool-down period was extended to eighteen (18) hours followed by storage at 5° C. (78%). The developed seeds stored at room temperature also resumed growth well (73%). For Dazzler® White, the treatment which caused the greatest loss in resumption of root elongation and root hair proliferation was storage at room temperature as only two percent (2%) continued growth within two days after sowing. In sharp contrast, the seeds cooled over eighteen (18) hours to 5° C. and then maintained at that temperature all (100%) resumed growth within 48 hours after sowing. Intermediate values for growth were noted when the seeds were directly placed into 5° C. (73%) or cooled down over six (6) hours and maintained at that temperature (43%). For both Impatiens Dazzler® Red and Dazzler® White, an eighteen-(18-) hour cool-down period followed by storage to 5° C. produced the most favorable results by maintaining the highest rates of resumptive root growth. These results serve to illustrate the importance of selecting the correct storage temperature for developed seeds and the critical importance of properly pre-conditioning the seeds for extended periods of cold storage.

TABLE 6

Impatiens Variety

| | Dazzler ® | Dazzler ® |
|---|---|---|
| Treatment | Red | White |
| 5° C. Direct | 12^{a} | 73 |
| 2 hours at 35° C. and to | 28 | 43 |
| 5° C. over 6 hours | | |
| 25° C. to 5° C. over 18 hours | 78 | 100 |
| Room temperature | 73 | 2 |

^{a}Percent developed seeds showing root elongation and root hair proliferation within two (2) days after sowing.

### EXAMPLE 9

### Post-Harvest Handling of Developed Seed: Storage

To demonstrate that developed seeds could be stored without loss of viability for a reasonable period following harvest, the following experiment was performed. Approximately 1,000 seeds of impatiens (varieties Dazzler® Cranberry and Super Elfin® White) were germinated in an aerated column containing 1L solution of 100 ppm Peters Fertilizer/100 ppm KNO₃/100 ppm AGRONOMIX®/0.5 mM citric acid/0.1 ppm NAA/7 ppm IBA/0.25 mM DTT at 25° C. in a lighted growth room. After 5 days in the germination solution, the precotyledon forms were harvested, gently centrifuged to remove the surface moisture, dried to 56% relative moisture, and subjected to one of three treatments.

The first treatment involved holding the precotyledon forms at 25° C. for 24 hours after harvesting, then slowly decreasing the temperature at a constant rate over 18 hours to a final temperature of 5° C. The precotyledon forms were then maintained at 5° C. for long-term storage. The second treatment involved simply cooling the precotyledon forms slowly at a constant rate over a period of 18 hours to a final temperature of 5° C. These precotyledon forms were also maintained at 5° C. for long-term storage. The third treatment was designed to test the idea that a heat-shock treatment (pulsed) could increase the shelf-life of the developed seed. With this in mind, the developed seed were raised to a temperature of 35° C. for 2 hours, quickly returned to 25° C., and then slowly cooled to 5° C. at a constant rate over a period of 18 hours. The developed seeds were then maintained at 5° C. for long-term storage. All developed seeds were sown the day after harvest, following the various post-harvest treatments and just prior to the start of the long-term storage at 5° C. The developed seeds were then sown again after four weeks of storage at 5° C.. For both sowings, 20 developed seeds of each variety were sown per treatment, and the number of developed seeds that grew into plants was scored 14 days after the date of sowing.

For all three treatments, no differences were detected at the beginning of the storage period (1 day after harvest) as the percentage of developed seeds which continued to develop into plants was 100%. In fact, no differences were detected at the end of the storage period (28 days post-harvest held at 5° C.) either as 100% of the sown precotyledon forms developed into plants. These results demonstrate that a number of different post-harvest treatments can be used to maintain the viability of the developed seeds.

The first treatment mimicked the situation in which harvested developed seeds would be shipped overnight at ambient temperature to growers for sowing. The second treatment demonstrated that cold acclimation directly after harvest also did not adversely affect the storage life of the precotyledon forms. Finally, the third treatment demonstrated that heat-shock treatments had no detrimental effect upon the developed seeds. These results demonstrate that a number of post-harvest treatments can be considered to maintain the viability of the precotyledon forms during storage, thus ensuring high yields of usable plants.

### EXAMPLE 10

### Early Photosynthetic Development and Enhanced Rooting in Impatiens Developed Seeds

To demonstrate that developed seeds, because of their advanced developmental state, give rise to seedlings (when sown in a suitable environment) that become photosynthetically-competent and photosynthetically-active earlier than raw seed sown in the same environment, the experiment described below was performed. At the same time, to gain a more comprehensive developmental profile of developed seed-derived seedlings, root area measurements were carried out on sown developed seeds selected from the same batch of developed seeds used for the photosynthetic measurements. As is shown in Table 8, these root measurements confirm results obtained in Example 7.

Approximately 1,000 seeds of impatiens (varieties Dazzler® Orange, Super Elfin® Cherry Improved, Super Elfin® Lavender, Super Elfin® Lilac, and Super Elfin® White, all commercially available from Ball Horticultural Company, 622 Town Road, West Chicago, Ill. 60185), were germinated in an aerated column containing a 1 L solution of 100 ppm Peters Fertilizer/100 ppm KNO₃/100 ppm AGRONOMIX®/0.5 mM citric acid/0.1 ppm NAA/2 ppm IBA/0.25 mM DTT at 25° C. in a lighted growth room. After 5 days in the germination solution, the precotyledon forms were harvested, sown in plug trays containing a soil-less seedling growing medium and maintained at 25° C. in a lighted (16 hours light and 8 hours dark) growth room. Raw seed from the same lot of these same varieties was also sown on the same day as the developed seed and maintained under identical conditions. After 2 days and 5 days, photosynthetic activity in the cotyledonary leaves of the developed seed- and raw seed-derived seedlings was determined using a Photosynthesis Yield Analyzer Mini-PAM manufactured by Heinz Walz GmbH (Germany). This fluorometer applies pulse-modulated measuring light for selective detection of chlorophyll fluorescence yield, which is a measure of photosynthetic activity. The results are shown below in Table 7.

TABLE 7

| Photosynthetic Activity | |
|---|---|
| Day 2 | Day 5 |

| Impatiens Variety | Developed | Raw | Developed | Raw |
|---|---|---|---|---|
| Dazzler ® Orange | 0.78 | ND² | 0.778 | 0.126 |
| | | | | (20)³ |
| Super Elfin ® | 0.752 | ND | 0.803 | 0.292 |
| Cherry Imp. | | | | (40) |
| Super Elfin ® | 0.774 | ND | 0.767 | 0.258 |
| Lavender | | | | (50) |
| Super Elfin ® | 0.764 | ND | 0.760 | 0.059 |
| Lilac | | | | (10) |
| Super Elfin ® | 0.780 | ND | 0.798 | 0.129 |
| White | | | | (20) |

¹Average photosynthetic yield from 10 seedlings.
²Not detected.
³Percent seedlings photosynthetically active.

As can be observed, all five impatiens varieties demonstrated similarly high levels of photosynthetic activity just 2 days after sowing of the developed seeds. All seedlings derived form the developed seeds exhibited easily detectable levels of photosynthetic activity. In sharp contrast, no photosynthetic activity could be measured in the raw seed-sown samples since they had not yet developed to the stage for photosynthesis to be detected. In fact, for nearly all varieties, no evidence of radicle protrusion could even be observed except in the case of some seeds of Super Elfin® Cherry Improved, and even in those instances, radicle protrusion was very slight.

Five days after sowing, photosynthetic activity remained high, as expected, in seedlings derived from developed seeds. However, photosynthetic activity still remained undetectable in a majority of the seedlings derived from raw seed. In fact, even in the variety showing the highest percentage of photosynthetically-active seedlings (Super Elfin® Lavender), only 50% of the seedlings had detectable levels of photosynthetic activity. Of the Super Elfin® Lilac seedlings derived from raw seed, only 10% showed detectable levels of photosynthetic activity. For all impatiens varieties, overall levels of photosynthetic activity remained sharply reduced in raw seed-derived seedlings as compared to those from developed seeds. These results convincingly demonstrate that developed seeds are photosynthetically active at a much earlier time after sowing than raw seed.

At the same time that the developed seeds and raw seeds were sown in plug trays, 20 of the precotyledon forms and 20 raw seeds of these same five varieties were sown on moistened blotter paper in germination boxes. These boxes were then maintained in a lighted growth chamber at 25° C. At three and six days after sowing (two days was not selected since no significant radicle protrusion had yet occurred in raw seeds), the impatiens seedlings were photographed with a CCD camera, and the total root area calculated using the software described in Example 7. These results are shown below in Table 8.

TABLE 8

| Root Area | |
|---|---|
| Day 3 | Day 6 |

| Impatiens Variety | Developed | Raw | Developed | Raw |
|---|---|---|---|---|
| Dazzler ® Orange | 215.4¹ | 3.6 | 313.4 | 94.3 |
| Super Elfin ® | 226.8 | 14.8 | 464.9 | 172.1 |
| Cherry Imp. | | | | |
| Super Elfin ® | 206.6 | 7.1 | 844.6 | 42.1 |
| Lavender | | | | |
| Super Elfin ® | 188.1 | 1.5 | 327.3 | 62.3 |
| Lilac | | | | |
| Super Elfin ® | 184.0 | 8.1 | 340.7 | 148.1 |
| White | | | | |

¹Cumulative total root area in mm².

As can be observed, by Day 3, all the impatiens seedlings derived from developed seed showed dramatically enhanced root proliferation compared to raw seed. For example, Dazzler® Orange precotyledon form-derived seedlings produced a total root area of 215.4 mm². By comparison, the Dazzler® Orange raw seed-derived seedlings yielded only 3.6 mm² of root tissue, only 1.7% of that observed for the developed seed samples. Similarly, the Super Elfin® Cherry Improved and Lavender raw seeds produced seedlings showing only 14.8 and 7.1 mm² of root tissue, respectively. In contrast, developed seeds of the same varieties generated 226.8 and 206.6 mm² of root tissue, respectively, representing increases of about 15- to about 30-fold. Finally, developed seed-derived seedlings of Super Elfin® Lilac and Super Elfin® White exhibited about 125- and about 23-fold increases, respectively, in root area over raw seed-derived seedlings. These results demonstrate that impatiens developed seeds exhibited from about 15- to about 125-fold more root area as compared to raw seed of the same varieties after just three days of growth.

On Day 6 after sowing, the seedlings derived from impatiens developed seed still exhibited more extensive root development than seedlings from raw seed. In fact, after six days growth, none of the raw seed-derived seedlings displayed root development that matched the developed seed seedlings after just three days growth. The best raw seed-rooting variety after six days, Super Elfin® Cherry Improved (172.1 mm²), still did not surpass the poorest developed seed-rooting variety, Super Elfin® White (184.0 mm²), measured on Day 3. In general, most of the impatiens seedlings grown from raw seed exhibited from about 3- to about 5-fold less root area than their developed seed counterparts, although germinated Super Elfin® Lavender raw seeds (42.1 mm²) displayed about 20-fold less root area than developed seeds of the same variety (844.6 mm²). These root area measurements (on both Day 3 and Day 6) demonstrate that impatiens developed seeds exhibit greatly enhanced root development compared to raw impatiens seed.

In conclusion, the photosynthetic rate and root area measurements demonstrate that impatiens developed seeds develop significantly faster than raw seed of impatiens after sowing. These results clearly support the observations that both root development and hypocotyl and cotyledonary leaf(s) development are enhanced in impatiens developed seeds (as compared to raw seed).

### EXAMPLE 11

### Post-Harvest Handling of Developed Seed: Multiple, Sequential Harvests

The following Example demonstrates that maximum yields of the precotyledon forms of developed seed can be obtained by; 1) placing the seeds in a germination environment containing the developed seed solution; 2) after a period of several days, harvesting and collecting as a purified fraction the precotyledon form of developed seed; 3) returning the remaining batch of seeds to the reaction vessel for additional treatment times; and 4) and again harvesting and collecting as a purified fraction the precotyledon form of developed seed. Steps 3 and 4 can be repeated multiple times to achieve maximum yields of developed seed.

Seeds of impatiens varieties Dazzler® Punch and Dazzler® Red were first primed in an osmotic solution containing PEG (−8 bar), and then placed into an aerated column containing a 1 L solution of 100 ppm Peters Fertilizer/100 ppm KNO₃/100 ppm AGRONOMIX®/0.5 mM citric acid/0.1 ppm NAA/4 ppm IBA/0.25 mM DTT (hereinafter the “Developed Seed Solution”) at 25° C. in a lighted growth chamber. When the first population of seeds had attained the desired precotyledon form of developed seed, the entire column of seeds was harvested and the developed seeds separated from the non-preferred seed forms (namely, non-germinated seeds) based upon their change in buoyant density by methods known in the art. The purified developed seed fraction was collected and the remaining seeds still requiring additional treatment time returned to the aerated solution for further treatment. Over the next four days, this procedure was repeated on a daily basis until nearly the entire batch of seeds had been harvested and collected as a purified developed seed fraction. The yield of developed seed for each day is shown below in Table 9. At the time of the first harvest (Day 1), only approximately 75% of the input seeds had attained the desired precotyledon form. Approximately 25% of the remaining seeds not collected were returned to the aerated developed seed solution for another day. On Day 2, the additional 24 hours of treatment increased the yields to 96% for both Dazzler® varieties. The remaining 4% of seeds were treated for an additional day, after which the yields increased to 99% for both Dazzler® varieties. By Days 4 and 5, the yields had increased to greater than 99% for both varieties. Thereupon, the ability to carry out multiple, sequential harvests over the time course of four to five days permitted the recovery of greater than 99% of the input seed, which is a critical prerequisite for the commercial implementation of the developed seed technology.

TABLE 9

| | Harvest Days | | | | |
|---|---|---|---|---|---|
| Impatiens Variety | Day 1 | Day 2 | Day 3 | Day 4 | Day 5 |
| Dazzlers ® Punch | 74^{a} | 96 | 99 | >99 | >99 |
| Dazzler ® Red | 78 | 96 | 99 | >99 | >99 |

^{a}Percent yield (number of developed seeds harvested/number input seeds)

### EXAMPLE 12

### Post-Harvest Handling of Developed Seeds: Purification and Enrichment

The following Example demonstrates that the precotyledon form of developed seed can be harvested and separated from other seeds (e.g., non-germinated seeds or germinating seeds requiring additional treatment times in the developed seed solution) by physical methods known in the art to yield a purified fraction.

Impatiens Dazzler® Red seeds were first primed in an osmotic solution containing PEG (−8 bar), and then placed into an aerated column containing the Developed Seed Solution at 25° C. in a lighted growth chamber. The developed seeds were harvested and slowly cooled to 5° C. over eighteen (18) hours prior to being sown (storage temperature was also 5° C.). On the first day of harvest (Day 1), a portion of the batch of seeds was randomly collected prior to density-based purification, placed into sealable plastic vials and cooled to 5° C. After separation and collection of the purified developed seed fraction, the developed seeds were treated identically as the non-separated ones. Both samples of seeds (three replicates of twenty (20) seeds each) were sown in germination boxes and after two days, the seeds scored for continued growth and development as measured by root elongation and the appearance of root hairs. This process was repeated on each of four (4) consecutive days. As shown below in Table 10 for Day 1, only twenty-two percent (22%) of the non-separated seeds displayed elongating roots and a proliferation of root hairs (within two (2) days) as compared to seventy-eight percent (78%) of the seeds present in the separated and purified fraction of developed seeds. Similarly, on Day 2 of harvesting, seventy-seven percent (77%) of the separated (and enriched) seeds showed evidence of growth within two (2) days, while only thirty-five percent (35%) of the seeds present in the non-separated fraction were similarly scored. For Days 3-5, the purified developed seed fraction all showed eighty-five percent (85%) to ninety-seven percent (97%) growth within two (2) days as compared to sixty-two percent (62%) to eighty-five percent (85%) of the seeds in the non-separated fraction. When all the days are taken together, the purified fraction of the developed seeds collected on each day of harvest consistently outperformed (showed higher rates of continued growth) the non-separated seed samples. By way of comparison, the primed Dazzler® Red seeds used as the starting material for this Example did not show signs of germination (namely, penetration of the seed coat by the radicle) until four days after sowing in the germination boxes. This comparison serves to illustrate that even after separation and purification, the developed seeds maintained their rapid pace of seedling development compared to primed seed.

TABLE 10

| Seed Population | Day 1 | Day 2 | Day 3 | Day 4 | Day 5 |
|---|---|---|---|---|---|
| Non-Separated | 22^{a} | 35 | 62 | 63 | 85 |
| Separated | 78 | 77 | 97 | 85 | 95 |

^{a}Percent seeds showing root elongation and root hair proliferation within 2 days after sowing.

### EXAMPLE 13

### Post-Harvest Handling of Developed Seed: Control of Relative Moisture Content

Impatiens Dazzler® Red developed seeds obtained as described in Examples 11 and 12 above, were harvested, separated, and rinsed with water. The separated developed seeds were subdivided into small amounts and vacuumed for different lengths of time (from no time to less than one minute) to achieve the different levels of moisture content. For the high treatment (in terms of relative moisture), the seeds showed a relative moisture content of about seventy-two percent (72%). For the low treatment (in terms of relative moisture), the moisture level was reduced to fifty-one percent (51%) relative moisture. For the medium treatment (in terms of relative moisture), the Dazzler® Red developed seeds had a value of about sixty percent (60%). These seeds were then slowly cooled to 5° C. over a period of 18 hours. A portion of the developed seeds (three replicates of 20 seeds each) was then sown in germination boxes and the developed seeds scored on Day 2 for continued plant development (as defined by root elongation and the appearance of root hairs). For the high treatment, only forty percent (40%) (24/60) (See Table 11 below) of the developed seeds showed evidence of continued growth within two (2) days of sowing. None (0/60) of the seeds subjected to the low treatment (See Table 11 below) showed evidence of root elongation and proliferation of root hairs. In sharp contrast, seventy-eight percent (78%) of the developed seeds subjected to the medium treatment (see Table 11 below) showed evidence of rapid root elongation and proliferation of root hairs. These results strongly indicate that the precotyledon form of developed seed is sensitive to moisture content and also, that the precotyledon form of developed seed is not desiccation-tolerant, but remains very sensitive to moisture conditions during post-harvesting handling steps.

TABLE 11

| Sample | Moisture Content | % Growth |
|---|---|---|
| High Moisture | 72% | 40 |
| Medium Moisture | 60% | 78 |
| Low Moisture | 51% | Zero |

### EXAMPLE 14

### Developed Seed: Seed Form Linked to an Increase in a Developmentally-Programmed Protein

A commercially available mouse monoclonal antibody prepared against human Cpn60 by StressGen Biotechnologies Inc. (British Columbia, Canada) was used that would be expected to recognize a highly conserved epitope in both the plastid and mitochondrial forms of plant Cpn60. The epitope, found at residues 383-419 of human Cpn60 shares a high degree of homology with the mitochondrial Cpn60 from Arabidopsis, rye, wheat, maize and winter squash. This highly-conserved epitope was also found in the plastid Cpn60 from Arabidopsis and spinach. It was empirically determined that the mouse monoclonal antibody reacted with impatiens mitochondrial and/or plastid Cpn60 protein using an enzyme linked immunosorbent assay (ELISA) test.

To determine whether Cpn60 protein levels increased during the methods used to generate developed seed, the following experiment was performed. Specifically, raw seeds from Impatiens variety Dazzler® Pink were primed in a high osmoticum environment containing PEG (−8 bar). The primed seeds were then placed into an aerated column containing the Developed Seed Solution at 25° C. in a lighted growth chamber. During this process, samples of seed were withdrawn from the aerated column at different time points and stored at −20° C. until all samples had been collected. The seed samples were removed on consecutive days from Day 3 to Day 7 after addition to the Developed Seed Solution. By Day 5, the impatiens seeds had developed to the pre-cotyledon form, and by Day 7, the cotyledon form of developed seed was observed.

Cell-free extracts were prepared from each of seven (7) samples (raw, primed and five developed seed samples from Day 3 to Day 7). The extracted proteins were bound to the walls of an ELISA plate well and the impatiens Cpn60 protein detected by sequential incubation with anti-Cpn60 antibody, a goat anti-mouse IgG antibody (conjugated to alkaline phosphatase), and finally, a substrate-containing reaction buffer that permitted color development. Purified human Cpn60 protein was used as a positive control.

The ELISA results showed that Cpn60 was present in low, but detectable amounts in dry impatiens seeds (see Table 12 below). After the priming treatment in PEG, the level of Cpn60 remained relatively unchanged. After three days incubation in the Developed Seed Solution, a twenty-percent (20%) increase in Cpn60 levels over raw seed was observed. The Cpn60 levels continued to rise on Days 4 and 5 to levels of thirty-six percent (36%) and fifty percent (50%) greater, respectively, than levels in raw seed. On Days 6 and 7, Cpn60 levels increased even further to amounts of seventy-eight percent (78%) and one hundred and twenty-two percent (122%) above raw seed levels, respectively. It should be especially noted that the developed seed samples assayed for Cpn60 levels on Day 5 (50% increase) and Day 7 (122% increase) most closely corresponded to the precotyledon and cotyledon forms of developed seed, respectively.

TABLE 12

| Impatiens Sample | Treatment | OD₄₀₅ |
|---|---|---|
| Dazzler ® Pink | Raw | 0.163^{a} |
| | Primed | 0.169 |
| | Developed - Day 3 | 0.194 |
| | Developed - Day 4 | 0.221 |
| | Developed - Day 5^{b} | 0.246 |
| | Developed - Day 6 | 0.291 |
| | Developed - Day 7^{C} | 0.363 |
| Impulse Apple Blossom | Pregerminated | 0.159 |

^{a}Absorbance units; ^{b}Precotyledon form; ^{c}Cotyledon form.

To demonstrate that the increases in Cpn60 protein levels in the precotyledon and cotyledon forms of developed seed were developmentally regulated (and not a result of the Developed Seed Solution per se), the levels of Cpn60 in impatiens seeds germinated in water rather than the Developed Seed Solution were determined. Primed impatiens seeds (variety Dazzler® Pink) were germinated in an aerated water column under the same environmental conditions used to create developed seeds. On Days 3 to 6, samples were withdrawn from the aerated column and cell-free extracts prepared from the tissues. The Cpn60 levels were then determined by ELISA as described above (see Table 13 below).

As will be seen, on Days 3 and 4, the Cpn60 levels increased to levels of fifteen percent (15%) and twenty-three percent (23%), respectively, above the levels found in the starting primed seed. Cpn60 levels increased even further on Days 5 and 6, finally reaching a level of seventy-eight percent (78%) above the level found in primed seed. These increases in Cpn60 levels are in general agreement with those measured for the developed seed forms (namely, the precotyledon and cotyledon forms). Taken together, these findings demonstrate that the rise in impatiens Cpn60 levels are not directly caused by chemical components in the developed seed solution, but rather are following the normal developmentally-regulated expression pattern observed in germinating impatiens seeds.

To assess whether Cpn60 levels increased in the developed seeds of other plant species, an ELISA using pansy protein extracts was conducted. For this experiment, the precotyledon and cotyledon forms of pansy developed seed were created from primed pansy seeds. The results (shown in Table 13 below), showed that Cpn60 protein levels increased 2.4-fold and 5.0-fold in pansy precotyledon and cotyledon forms, respectively, compared to primed pansy seed. These increases were larger than those observed in impatiens developed seed, and are consistent with the idea that Cpn60 protein levels in germinating pansy seeds are also tightly regulated at the developmental level.

In a further experiment, the Cpn60 levels in pregerminated impatiens seeds were examined. Pregerminated seeds have been described as being more developmentally advanced than either raw or primed seed because the radicle has penetrated the seed coat and extends out a very short distance. The purpose of this experiment was to determine if this seed form would be considered developmentally-advanced based upon the level of Cpn60 protein in the pregerminated seed. A sample of PreMagic impatiens seeds (Impulse Apple Blossom, commercially available from Novartis Seed, Inc. Flowers, 5300 Katrine Avenue, Downers Grove, Ill. 60515), was obtained and the Cpn60 level determined in this seed. It was found that the level of Cpn60 in PreMagic impatiens seeds was virtually identical to the levels found in raw (untreated) and primed Dazzler® Pink impatiens seeds (See Table 12).

Thus, although the pregerminated seeds are more developmentally advanced than either raw or primed seed (based upon visualization of the protruding radicle), these results suggest that they are only marginally so since the Cpn60 content remains at a basal level. These results clearly demonstrate that pregerminated seeds are much less developmentally advanced than the precotyledon and cotyledon forms of developed seeds (see also Example 2 wherein pregerminated pansy seeds were used as the starting material for obtaining the precotyledon and cotyledon forms of pansy developed seed). Taken altogether, these results clearly demonstrate that the precotyledon and cotyledon forms of impatiens and pansy developed seed contain elevated levels of Cpn60 protein, a protein whose synthesis is developmentally-regulated during the seed germination process and seedling establishment in several species, including impatiens. No significant increase in Cpn60 levels could be detected in neither primed nor pregerminated impatiens seed, an observation consistent with the relatively early developmental stage (as compared to developed seed) achieved in these enhanced seed products.

TABLE 13

| Sample | Treatment | OD₄₀₅ |
|---|---|---|
| Impatiens Dazzler ® Pink | Raw | 0.136^{a} |
| | Water - Day 3 | 0.156 |
| | Water - Day 4 | 0.167 |
| | Water - Day 5 | 0.168 |
| | Water - Day 6 | 0.242 |
| Pansy Bingo White | Primed | 0.092 |
| | Developed - Precotyledon | 0.222 |
| | Developed - Cotyledon | 0.465 |

^{a}Absorbance units.

### EXAMPLE 15

### Desiccation-Tolerant Developed Seed

Example 13 demonstrated that the precotyledon form of impatiens developed seed was sensitive to relative moisture content during post-harvest handling procedures, and that the reduced relative moisture content was detrimental to the continued growth of the developed seed after being sown in a suitable environment. U.S. Pat. No. 5,522,907 describes methods for inducing desiccation tolerance in the radicles of pregerminated seeds, specifically impatiens pregerminated seeds.

A study was conducted to determine if the methods for inducing desiccation tolerance in the radicles of pregerminated seeds could be similarly applied to induce desiccation tolerance in the modified root structure of developed seeds. In this study, the precotyledon forms of impatiens and vinca developed seeds were created essentially as described in Example 4, and then subjected to various post-harvest protocols in an attempt to induce desiccation tolerance.

For impatiens, developed seed of varieties Super Elfin® Pink Swirl, Stardust Pink, and Dazzler® Rose (all available from Ball Horticultural Company, West Chicago, Ill.) were created and stored at 5° C. for 24 hours. After this time, the impatiens developed seeds for each variety were divided into three portions, and each portion was then subjected to one of three post-harvest treatments.

One portion of the impatiens developed seeds was placed on blotting paper saturated with PEG-8000 (324 g/L) having a water potential of −1.5 MPa, and incubated at 8° C. in the dark for a period of 6 days. After this treatment, the seeds were thoroughly rinsed, blotted dry and then exposed to conditions of 40% relative humidity and 20° C. to reduce the moisture content of the developed seed to about 4% to about 12% within 24 hours.

A second portion of the impatiens developed seeds was stored in a sealed container and incubated at 8° C. in the dark for a period of 6 days (during the same time period as the PEG-8000-treated developed seeds). After this period, these developed seeds were exposed to conditions of 40% relative humidity and 20° C. to reduce the moisture content of the developed seed to 4-12% within 24 hours.

A third and final portion of the impatiens developed seeds was permitted to remain in sealed containers at 5° C. in the dark for a period of 7 days, while the other developed seed treatments were ongoing.

At the conclusion of all three treatments, the relative moisture contents of the impatiens developed seeds subjected to each protocol were determined (using only a fraction of the seeds). Finally, remaining seeds were sown in germination boxes (25 per box) containing moistened blotter paper and incubated in the light at 25° C. for a period of 14 days to permit growth of the developed seeds, at which time the developed seeds were evaluated for continued growth.

As can be observed in Table 14, the Super Elfin® Pink Swirl and Stardust Pink varieties that were maintained at 5° C. in sealed containers throughout the course of the study had relative moisture contents of 36% and 46%, respectively. Similarly, Dazzler® Rose developed seeds stored under the same environmental conditions had a relative moisture content of 36%. When sown, 100% of these impatiens developed seeds continued growth. Impatiens exhibit seminal root growth.

The developed impatiens seeds that were subjected to 6 days of incubation at 8° C. in the dark followed by drying conditions of 40% relative humidity and 20° C. were measured to have relative moisture contents of 6%-9%. When the impatiens developed seeds subjected to this treatment were sown, dramatically reduced rates of continued growth were observed. Super Elfin® Pink Swirl and Stardust Pink varieties both had only 48% of the developed seeds continue growth, whereas Dazzler® Rose developed seeds were slightly higher at 64%. These values stand in sharp contrast to the 100% values that were noted for the developed seeds of all three varieties that were stored at 5° C. and were found to have relative moisture contents of 36-46%.

Finally, the developed seeds that were subjected to PEG-8000 treatment followed by exposure to conditions of 40% relative humidity and 20° C. were measured to have relative moisture contents of 6-9%, essentially the same as was noted for the developed seeds that were dried down, but without first being exposed to PEG-8000. When the PEG-8000-treated (and dried) seeds were sown and incubated as described above, high percentages of growth were observed for all three varieties. For Dazzler® Rose, 88% of the sown developed seeds continued growth. For the remaining two varieties, the percentages were even higher as 92% and 100% of the Super Elfin® Pink Swirl and Stardust Pink developed seeds, respectively, continued growth after being sown. These results indicate that impatiens developed seeds can be dried down to form desiccation-tolerant developed seed with relative moisture levels that are typical of those found for non-germinated seed, and still continue growth.

Furthermore, these results illustrate that the some, but not all, of the desiccation-tolerance induction treatments for pregerminated seed described in U.S. Pat. No. 5,522,907 can be similarly applied to impatiens developed seed (precotyledon form) to achieve the same results. The PEG-8000-treated (and dried) impatiens developed seeds (for all varieties), when sown, demonstrated continued growth rates of 88%-100%, as compared to only 48%-64% for the developed seeds not treated with PEG-8000 that were dried to the same relative moisture content (6%-9%). That is, the impatiens developed seeds stored at 8° C. in the dark for 6 days do not gain desiccation tolerance by this route and subsequently cannot withstand well the drying conditions used here (40% relative humidity and 20° C. for 24 hours).

It should be noted that the conditions used here (8° C. for 6 days), in which the impatiens developed seed was not able to gain desiccation tolerance, are quite similar to the conditions described in U.S. Pat. No. 5,522,907 (8° C. for 5 days) where desiccation tolerance was able to be induced in pregerminated impatiens seeds. In that U.S. patent, those inventors reported that pregerminated impatiens seeds incubated at 8° C. for 5 days in sealed containers were able to withstand even very fast drying conditions (reaching 5% relative moisture in just 6 hours after being exposed to 30% relative humidity and 20° C.) and continue growth at very high rates (96%).

In sharp contrast, the impatiens developed seeds treated similarly here (8° C. for 6 days followed by exposure to 40% relative humidity and 20° C. for 24 hours) were only able to continue growth at rates between 48% and 64%. However, as was also demonstrated, impatiens developed seeds can be treated so that they can withstand further drying conditions that reduce their relative moisture content to 6%-9%, and still retain their ability to continue growth at very high percentages.

These observations taken together further support the conclusions drawn in Examples 2 and 14 that impatiens pregerminated seeds and developed seeds differ in their final developmental stages achieved. The distinction here is based upon the ability of pregerminated seeds and the inability of developed seeds to gain desiccation tolerance after being exposed to the same environmental conditions (5-6 days at 8° C.).

The above-described three post-harvest protocols applied to impatiens developed seed were similarly applied to the precotyledon form of vinca developed seed to determine if desiccation tolerance could also be induced in this ornamental species. Vinca exhibits non-seminal root growth.

For these studies, developed seed was created for two varieties of vinca, Cooler Raspberry Red and Cooler Orchid (both available from Ball Horticultural Company, West Chicago, Ill.). The vinca developed seed was then divided into three portions and treated as described above for the impatiens developed seed. The two portions incubated at 8° C. for 6 days were then dried down under conditions of 40% relative humidity and 20° C. for 24 hours to achieve final relative moisture contents of 8-10%. All vinca developed seeds were then sown on moistened blotter paper, incubated at 25° C. in the light, and evaluated after 14 days of incubation as described for impatiens developed seeds.

The vinca developed seeds stored and maintained at 5° C. for both Cooler Raspberry Red and Cooler Orchid varieties continued growth at 100% levels, indicating that seed viability was well maintained in vinca developed seed containing 32%-36% relative moisture content. This result is in excellent agreement with what was observed for the impatiens developed seed that was treated similarly (all impatiens developed seeds continued growth after sowing in a suitable environment).

When the vinca developed seeds that were dried back to a relative moisture content of 8%-9% after being held for 6 days at 8° C. were examined for growth, it was observed that none (zero %) of each of the Cooler Raspberry Red and Cooler Orchid developed seeds continued growth. Moreover, even those vinca developed seeds that were first treated with PEG-8000 for 6 days at 8° C. to in an attempt to induce desiccation tolerance were unable to grow after being dried back to 8%-9% relative moisture. That is, once again, none (zero %) of the vinca developed seeds treated with PEG-8000 and then dried down were able to continue growth.

These results strongly support the conclusion that the desiccation tolerance induction methods described in U.S. Pat. No. 5,522,907 are ineffective for vinca developed seeds, when a moisture content of about 4% to about 12% is desired. Moreover, these vinca developed seed results taken together with those observed for impatiens developed seed strongly illustrate that the developed seeds of the present invention differ substantially from pregerminated seeds, this point being best illustrated here by the variable responses exhibited by developed seeds of impatiens and vinca in their response to desiccation-tolerance induction protocols. These observations and results also serve to illustrate that the developed seeds of the present invention are best characterized as being not desiccation-tolerant, but in some instances, can be induced to be desiccation-tolerant after being subjected to an effective desiccation-tolerance induction protocol.

TABLE 14
Desiccation induction treatments
for impatiens and vinca developed seeds

| | | | Relative | Percent¹ |
|---|---|---|---|---|
| Species | Variety | Treatment | Moisture | Growth |
| Impatiens | | | | |
| | Pink Swirl | | | |
| | | Control | 36 | 100 |
| | | −PEG | 9 | 48 |
| | | +PEG | 9 | 92 |
| | Stardust Pink | | | |
| | | Control | 46 | 100 |
| | | −PEG | 6 | 48 |
| | | +PEG | 7 | 100 |
| | Rose | | | |
| | | Control | 36 | 100 |
| | | −PEG | 7 | 64 |
| | | +PEG | 6 | 88 |
| Vinca | | | | |
| | Raspberry Red | | | |
| | | Control | 36 | 100 |
| | | −PEG | 9 | Zero |
| | | +PEG | 9 | Zero |
| | Orchid | | | |
| | | Control | 32 | 100 |
| | | −PEG | 8 | Zero |
| | | +PEG | 8 | Zero |

¹Percent of developed seeds showing continued growth

Each of the patents, applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art. Changes can be made to the composition, operation and arrangement of the method of the present invention described herein without departing from the concept and scope of the invention as defined in the following claims.

What is claimed is:

1. A developed seed that is singulated and free-flowing and can be operationally sown in the same manner as raw, primed or pregerminated seed, said developed seed comprising a modified root structure and having its root development interrupted and altered, said developed seed exhibiting an emerged hypocotyl or lacking an emerged hypocotyl, wherein said root development is capable of resuming when the developed seed is sown in a suitable environment, and further wherein said developed seed is capable of developing into a usable plant(s) when sown in a suitable environment.

2. The developed seed of claim 1 wherein said developed seed exhibits higher levels of chaperonin 60 when compared to raw, primed or pregerminated seed.

3. The developed seed of claim 1 that exhibits an emerged hypocotyl.

4. The developed seed of claim 1 wherein the developed seed further exhibits enhanced rooting when compared to raw, primed or pregerminated seed.

5. The developed seed of claim 1 wherein the developed seed further exhibits earlier photosynthetic activity when compared to raw, primed or pregerminated seed.

6. The developed seed of claim 1 wherein the developed seed is from plants selected from the group consisting of Alliums, Antirrhinums, Asteraceae, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Violas, Apiums, Daucuses, Chicoriums and Zinnias.

7. The developed seed of claim 1 further comprising a seed coating.

8. A plant grown from the developed seed of claim 1.

9. The plant of claim 8 having a shorter internode length than a plant grown from raw, primed or pregerminated seed.

10. The developed seed of claim 1 that is desiccation-intolerant.

11. A developed seed that is singulated and free-flowing and can be operationally sown in the same manner as raw, primed or pregerminated seed, said developed seed comprising a modified root structure and having its root development interrupted and altered, said developed seed exhibiting an emerged hypocotyl or lacking an emerged hypocotyl, wherein said root development is capable of resuming when the developed seed is sown in a suitable environment, and further wherein said developed seed exhibits higher levels of chaperonin 60 when compared to raw, primed or pregerminated seed and further wherein said developed seed is capable of developing into a usable plant(s) when sown in a suitable environment.

12. The developed seed of claim 11 comprising an emerged hypocotyl.

13. The developed seed of claim 11 wherein the developed seed further exhibits enhanced rooting when compared to raw, primed or pregerminated seed.

14. The developed seed of claim 11 wherein the developed seed further exhibits earlier photosynthetic activity when compared to raw, primed or pregerminated seed.

15. The developed seed of claim 11 further comprising a seed coating.

16. The developed seed of claim 11 wherein the developed seed is from plants selected from the group consisting of Alliums, Antirrhinums, Asteraceae, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Catharanthus, Violas, Apiums, Daucuses, Chicoriums and Zinnias.

17. A plant grown from the developed seed of claim 11.

18. The plant of claim 17 having a shorter internode length than a plant grown from raw, primed or pregerminated seed.

19. The developed seed of claim 11 that is desiccation-intolerant.

20. A developed seed having higher levels of chaperonin 60 when compared to raw, primed or pregerminated seed and which is capable of developing into a usable plant(s) when sown in a suitable environment.

21. The developed seed of claim 20 wherein said developed seed comprises a modified root structure and has its root development interrupted and altered, said developed seed exhibiting an emerged hypocotyl or lacking an emerged hypocotyl, wherein said root development is capable or resuming when the developed seed is sown in a suitable environment.

22. The developed seed of claim 21 comprising an emerged hypocotyl.

23. The developed seed of claim 20 wherein the developed seed further exhibits enhanced rooting when compared to raw, primed or pregerminated seed.

24. The developed seed of claim 20 wherein the developed seed further exhibits earlier photosynthetic activity when compared to raw, primed or pregerminated seed.

25. The developed seed of claim 20 further comprising a seed coating.

26. The developed seed of claim 20 wherein the developed seed is from plants selected from the group consisting of Alliums, Antirrhinums, Asteraceae, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Violas, Apiums, Daucuses, Chicoriums and Zinnias.

27. The developed seed of claim 20 that is desiccation-intolerant.

28. A plant grown from the developed seed of claim 20.

29. The plant of claim 28 having a shorter internode length than a plant grown from raw, primed or pregerminated seed.

30. A process of making developmentally advanced developed seed having its root development interrupted and altered, wherein said root development is capable of resuming when the developed seed is sown in a suitable environment, the process comprising the steps of:
(a) placing a batch of seed or somatic embryos in a germination environment comprising at least one auxin; and
(b) maintaining the seed or somatic embryos in the germination environment a time period sufficient to form a precotyledon form to the developed seed.

31. The process of claims 30 wherein the germination environment contains nutrients.

32. The process of claim 30 wherein the germination environment contains at least one organic acid.

33. The process of claim 30 further comprising the steps of:
(c) separating a developed seed fraction from seed or somatic embryos that had not formed developed seed; and
(d) collecting a purified developed seed fraction.

34. The process of claim 33 further comprising the step of:
(e) maintaining said seed or somatic embryos that had not formed developed seed in said germination environment until a precotyledon form of the developed seed is obtained.

35. The process of claim 33 further comprising the step of removing the residual external moisture from the surface of the developed seed.

36. The process of claim 33 further comprising the step of cooling the developed seed over a period of from about 6 to about 20 hours to a temperature of from about 1° C. to about 15° C.

37. The process of claim 36 wherein the developed seed is stored at a temperature of from about 1° C. to about 15° C.

38. The process of claim 30 wherein the developed seed is from plants selected from the group consisting of Alliums, Antirrhinums, Begonias, Brassicaceae, Capsicums, Betas, Lycopersicons, Cucurbitaceae, Cyclamens, Dianthuses, Gazanias, Gerberas, Impatiens, Lisianthus, Lobelias, Matthiolas, Nicotianas, Pelargoniums, Petunias, Phloxes, Poaceae, Primulas, Raphanuses, Salvias, Solanaceae, Tagetes, Turfgrass, Verbenas, Catharanthus, Violas, Apiums, Daucuses, Chicoriums and Zinnias.

39. The process of claim 33 including the further step of forming desiccation-tolerant developed seed that contains about 4 to about 30 percent moisture.

40. The process of claim 39 wherein the seed exhibits seminal growth.

41. The process of claim 40 wherein the developed seed has a moisture content of about 4 to about 12 percent.

42. A developed seed produced by the process of claim 30.

43. A plant grown from the developed seed of claim 42.
